(19) United States
(12) Patent Application Publication
(10) Pub. No.: US 2012/0016167 A1
(43) Pub. Date: Jan. 19, 2012

(54) HYDROPROCESSING OF BIOCOMPONENT FEEDS WITH LOW PRESSURE HYDROGEN-CONTAINING STREAMS

(75) Inventor: Patrick L. Hanks, Annandale, NJ (US)

(73) Assignee: EXXONMOBIL RESEARCH AND ENGINEERING COMPANY, Annandale, NJ (US)

(21) Appl. No.: 12/836,924

(22) Filed: Jul. 15, 2010

(51) Int. Cl. C10L 1/16; C07C 1/00

(52) U.S. Cl. 585/14; 585/733; 585/310

(57) ABSTRACT

The invention relates to a method for forming an at least partially renewable diesel product, comprising: hydrodeoxygenating a feedstock comprising about 0.1 wt % to about 50 wt % of a fresh biocomponent portion with a relatively pure hydrogen treat gas in the presence of a hydrodeoxygenation catalyst under relatively low total pressure to form a hydrodeoxygenated product; and separating the hydrodeoxygenated product into a vapor effluent and a liquid effluent, at least a first portion of the latter being recycled to the hydrodeoxygenation step, and at least a second portion of the latter being a diesel product. Advantageously, the hydrodeoxygenation catalyst can have a relatively low catalytic activity (about 50% or less of fresh HDS/HDN activity).

### FIELD

Systems and processes are provided for catalytic hydrotreatment of biocomponent feeds using a relatively low pressure, relatively high purity hydrogen source.

### BACKGROUND

Fuels based on biocomponent sources will likely become increasingly prevalent in the future. Already, various governments have instituted current and future requirements that motor fuel pools contain a minimum percentage of fuel derived from a biocomponent source, such as a plant, animal, fish, or algae-based oil or fat.

Producing diesel fuel from biocomponent sources also presents a variety of challenges. In particular, for diesel hydroprocessing units that operate at low pressures, the presence of the additional heteroatoms in a biocomponent based diesel feed may pose difficulties. Modifying and/or replacing low pressure units to allow for higher processing pressures would require expensive capital investment.

For production of diesel fuel, vegetable oils such as canola oil, palm oil, or other similar oils have been identified as potentially suitable based on the carbon chain length of the vegetable oil. While some progress has been made toward stand alone processing and/or co-processing of biocomponent feeds, improvements to allow efficient processing in a refinery setting are greatly desired.

U.S. Patent Application Publication No. 2008/0154073 describes a process for removing oxygen from biocomponent molecules at low hydrogen pressure. The feed in this reference is exposed to a supported hydrogenation catalyst, such as Ni, NiMo, Pt, or Pd, in the presence of 150-290 psi hydrogen.

U.S. Patent Application Publication No. 2008/0161614 describes two stage co-processing of a feed including both vegetable/animal and mineral oil. According to this disclosure, the first stage is operated at lower severity to primarily treat the vegetable and/or animal oil in the feed. The product of the first stage is then stripped to remove gas phase impurities. The stripped product is then hydrotreated in a more severe hydrotreatment stage to produce a diesel fuel.

International Publication No. WO 2008/040980 describes reducing hydrogen consumption by controlling the products from reactions to remove oxygen from biocomponent feeds. Lower hydrogen pressures are mentioned as helping to reduce hydrogen consumption, but such pressures are mentioned as also leading to catalyst deactivation.

International Publication No. WO 2008/020048 describes a process for production of normal alkanes by hydrotreating mixtures of triglycerides or free fatty acids with vacuum gasoil. Conventional hydrotreatment catalysts are used in this process, which takes place at relatively mild conditions including a reaction pressure below 100 bars.

European Patent No. EP 1719811 describes a method for producing liquid hydrocarbons from biomass. The method includes forming an aqueous slurry of the biomass and particles of a layered catalyst, such as a clay. The slurry is heated to a temperature of 250-400° C. Up to 10 bars of hydrogen may optionally be added, although the publication indicates a preference to perform the process without added hydrogen.

European Patent No. EP 1741767 describes a process for producing diesel fuel from biocomponent sources. This reference states that the process reduces the needed hydrogen consumption by adding sulfur-containing compound to the biocomponent feed.

European Patent No. EP 1693432 describes co-processing of vegetable oils with various diesel type mineral refinery feeds. The method appears to include combining vegetable oil and mineral oil, hydrotreating the combined oil, and stripping off gas phase products.

### SUMMARY

One aspect of the invention relates to a method for forming a biocomponent-containing diesel boiling range product, comprising: hydrodeoxygenating a feedstock comprised of about 50 wt % or less of a fresh biocomponent portion and having an oxygen content of at least 2 wt % by contacting the feedstock with a treat gas comprising at least about 80 mol % hydrogen in the presence of a hydrodeoxygenation catalyst under hydrodeoxygenation conditions to form a hydrodeoxygenated product; and separating the hydrodeoxygenated product into a liquid effluent and a vapor effluent, at least a first portion of the liquid effluent having an oxygen content of about 500 wppm or less being recycled to the hydrodeoxygenation step, and at least a second portion of the liquid effluent having an oxygen content of about 500 wppm or less being a diesel boiling range product, wherein the hydrodeoxygenation conditions comprise a total pressure from about 80 psig (about 0.5 MPag) to about 400 psig (about 2.8 MPag), and wherein the hydrodeoxygenation catalyst comprises a catalyst having an activity that is about 50% or less of an activity of a fresh hydrotreating catalyst having hydrodenitrogenation and/or hydrodesulfurization functionality.

### BRIEF DESCRIPTION OF THE DRAWINGS

FIG. 1 schematically shows a reaction system for performing a process according to an embodiment of the invention.

FIG. 2 schematically shows a reaction system for performing a process according to an embodiment of the invention.

FIG. 3 schematically shows a reaction system for performing a process according to an embodiment of the invention.

FIG. 4 schematically shows a reaction system for performing a process according to an embodiment of the invention.

FIG. 5 schematically shows a reaction system for performing a process according to an embodiment of the invention.

FIG. 6 schematically shows a reaction system for performing a process according to an embodiment of the invention.

FIG. 7 schematically shows a reaction system for performing a process according to an embodiment of the invention.

### DETAILED DESCRIPTION OF THE EMBODIMENTS

Building new hydroprocessing units for wholly renewable feeds in and/or incorporating new functionality for hydroprocessing feeds containing renewable components into a refinery can present many challenges. There is the obvious cost component of introducing new hardware, but adapting wholly mineral oil-based hydroprocessing units for co-processing of biocomponent-containing feeds can present its own challenges.

The oxygen content in a biocomponent feed can create a variety of problems in a refinery. The oxygen content can be as high as about 10-12% by weight, meaning that a large amount of hydrogen may be needed to remove the oxygen by a hydrogenation type reaction. Biocomponent feeds can also have a tendency to suppress hydrodesulfurization (HDS) activity and/or hydrodenitrogenation (HDN) activity of typical hydrotreating catalysts (e.g., Group VIB/Group VIII supported and/or bulk catalysts), making co-processing of a biocomponent feed challenging. However, the oxygen removal reaction can occur under relatively mild conditions and typically will not require relatively harsh conditions. Thus, a relatively low activity hydrogenation catalyst can not only be sufficient but may also be desirable to catalyze the hydrodeoxygenation (HDO) reaction. Similarly, relatively low hydrogen partial pressures can often be sufficient to perform HDO. By using a relatively low cost catalyst for hydroprocessing the biocomponent-containing feed, the invention allows for production of a biocomponent based fuel (e.g., diesel) product at reduced expense.

Additionally or alternately to effectively utilizing hydrogen resources, there may be an economic driver to use other refinery resources in such a way as to improve and/or maximize their effectiveness. For instance, at least partially spent catalyst (e.g., hydrotreating catalyst having an activity that is about 50% or less of an activity of a fresh catalyst, particularly one imparting HDN and/or HDS functionality) can be reused and/or existing reactors, particularly those reactors having a lower pressure capacity than desired, can be re-used in the present invention to improve and/or maximize the output efficiency of an already-constructed refinery.

In various embodiments, the invention allows a biocomponent feed to be hydrotreated with a relatively pure hydrogen source in order to remove the majority (i.e., more than 50% by weight) of the oxygen in the feed, and possibly substantially all (e.g., at least 95% by weight, preferably at least 98% by weight, for example at least 99% by weight, at least 99.5% by weight, at least 99.9% by weight, at least 99.95% by weight, at least 99.97% by weight, at least 99.98% by weight, at least 99.99% by weight, at least 99.995% by weight, or completely all) of the oxygen in the feed.

The costs for this process can be further reduced by using a relatively low cost water gas shift catalyst, such as Fe₃O₄, as the hydrotreatment catalyst. After processing, the resulting biocomponent product may be suitable for use, e.g., in the diesel fuel pool. Alternately, the biocomponent product can be mixed with a conventional mineral feed (e.g., kerosene, diesel, atmospheric gasoil, or the like, or a combination thereof), e.g., which can include a prior hydrotreatment step (but usually only for diesel and higher boiling range feeds; kerosene can generally be used without prior hydrotreatment, but may optionally undergo prior hydrotreatment if its sulfur and/or nitrogen contents are unsuitable for the HDO reaction). However, because the majority of the oxygen should be removed in a dedicated process, any co-processing of the biocomponent product with a mineral feed should typically have a reduced or minimized amount of catalyst suppression/deactivation problems.

In various embodiments, a system and method are provided for hydroprocessing a biocomponent feed. The system and method can include providing a HDO reactor for processing of a biocomponent feed. The catalyst for the hydrotreatment reactor can be an at least partially spent hydrotreating catalyst and/or a relatively low cost water gas shift catalyst that has sufficient HDO activity. The hydrogen source for the hydrotreatment reaction can include and/or be a relatively high purity gas source, e.g., containing hydrogen in an amount of at least about 80 mol %. In certain embodiments, steam can optionally also be introduced into the reactor to further facilitate hydrogen production via a water gas shift reaction. The hydrotreatment process can result in an effluent including a vapor product, optionally an aqueous product distinct from the vapor product, and a diesel boiling range hydrodeoxygenated product. Optionally, the diesel boiling range hydrodeoxygenated product can be co-processed with a mineral feed in a second hydrotreatment reactor. Optionally, the diesel boiling range hydrodeoxygenated product can be isomerized to improve the cold-flow properties. Optionally but preferably, the diesel boiling range hydrodeoxygenated product can be mixed, typically but not necessarily prior to separation, with another diesel boiling range stream, preferably either previously hydrotreated to attain a desired sulfur and/or nitrogen content or untreated and already having an acceptable sulfur and/or nitrogen content. Additionally or alternately, in such an embodiment, at least a portion of the diesel boiling range hydrodeoxygenated product, of the other diesel boiling range stream, or of the combined diesel boiling range stream, which exhibit desirable oxygen, sulfur, and nitrogen contents, can be recycled to the hydrodeoxygenation stage, optionally after having been catalytically hydroisomerized under appropriate conditions to improve cold-flow properties. Further additionally or alternately, in such an embodiment, at least a portion of the diesel boiling range hydrodeoxygenated product, of the other diesel boiling range stream, or of the combined diesel boiling range stream, which exhibit desirable oxygen, sulfur, and nitrogen contents, can be sent directly to the diesel pool, optionally after being stripped but without further hydroprocessing.

### Feedstocks

The feedstock can include varying amounts of feedstreams based on biocomponent sources. When desired, the feed can include at least about 0.1 wt % of feed based on a biocomponent source, for example at least about 0.5 wt %, at least about 1 wt %, at least about 3 wt %, at least about 5 wt %, at least about 10 wt %, or at least about 15 wt %. In such embodiments, the feed can include about 60 wt % or less of biocomponent, preferably about 50 wt % or less, for example about 40 wt % or less, or about 30 wt % or less. In other embodiments, the amount of biocomponent feed (e.g., for co-processing with the mineral oil portion of the feed) can be relatively small, for instance with a feed that includes at least about 0.5 wt % of feedstock based on a biocomponent source, e.g., at least about 1 wt %, at least about 2.5 wt %, or at least about 5 wt %. In such embodiments, the feed can include about 20 wt % or less of biocomponent based feedstock, for example about 15 wt % or less, about 10 wt % or less, or about 5 wt % or less.

As used herein, a “biocomponent feedstock” refers to a hydrocarbon feedstock (typically also containing some oxygen atoms) derived at least in part from a biological raw material component, such as vegetable fats/oils and/or animal fats/oils (including algae and fish fats/oils, respectively). Note that for the purposes of this document, vegetable fats/oils refer generally to any plant based material, and include fat/oils derived from a source such as plants from the genus Jatropha. Biocomponent feedstocks usable in the present invention can include any of those which comprise primarily triglycerides and free fatty acids (FFAs). The triglycerides and FFAs typically contain aliphatic hydrocarbon chains in their structure having from 8 to 36 carbons, preferably from 10 to 26 carbons, for example from 14 to 22 carbons. Types of triglycerides can be determined according to their fatty acid constituents. The fatty acid constituents can be readily determined using Gas Chromatography (GC) analysis. This analysis involves extracting the fat or oil, saponifying (hydrolyzing) the fat or oil, preparing an alkyl (e.g., methyl) ester of the saponified fat or oil, and determining the type of (methyl) ester using GC analysis. In one embodiment, a majority (i.e., greater than 50%) of the triglyceride present in the lipid material can be comprised of C₁₀ to C₂₆ fatty acid constituents, based on total triglyceride present in the lipid material. Further, a triglyceride is a molecule having a structure identical to the reaction product of glycerol and three fatty acids. Thus, although a triglyceride is described herein as being comprised of fatty acids, it should be understood that the fatty acid component does not necessarily contain a carboxylic acid hydrogen. In one embodiment, a majority of triglycerides present in the biocomponent feed can preferably be comprised of C₁₂ to C₁₈ fatty acid constituents, based on total triglyceride content. Other types of feed that are derived from biological raw material components can include fatty acid esters, such as fatty acid alkyl esters (e.g., FAME and/or FAEE).

Optionally, the feed can comprise a blend of a mineral oil feedstock with a biocomponent feedstock. By “mineral” or “mineral oil” feedstock is meant a fossil/mineral fuel source, such as crude oil, and not the commercial organic product, such as sold under CAS number 8020-83-5, e.g., by Aldrich. In the discussion below, a biocomponent feedstock refers to a hydrocarbon feedstock derived from a biological raw material component, from biocomponent sources such as vegetable, animal, fish, and/or algae. Generally, these biocomponent sources can include vegetable fats/oils, animal fats/oils, fish oils, pyrolysis oils, and algae lipids/oils, as well as components of such materials, and in some embodiments can specifically include one or more type of lipid compounds. Lipid compounds are typically biological compounds that are insoluble in water, but soluble in nonpolar (or fat) solvents. Non-limiting examples of such solvents include alcohols, ethers, chloroform, alkyl acetates, benzene, and combinations thereof.

Major classes of lipids include, but are not necessarily limited to, fatty acids, glycerol-derived lipids (including fats, oils and phospholipids), sphingosine-derived lipids (including ceramides, cerebrosides, gangliosides, and sphingomyelins), steroids and their derivatives, terpenes and their derivatives, fat-soluble vitamins, certain aromatic compounds, and long-chain alcohols and waxes.

In living organisms, lipids generally serve as the basis for cell membranes and as a form of fuel storage. Lipids can also be found conjugated with proteins or carbohydrates, such as in the form of lipoproteins and lipopolysaccharides.

Examples of vegetable oils that can be used in accordance with this invention include, but are not limited to rapeseed (canola) oil, soybean oil, coconut oil, sunflower oil, palm oil, palm kernel oil, peanut oil, linseed oil, tall oil, corn oil, castor oil, jatropha oil, jojoba oil, olive oil, flaxseed oil, camelina oil, safflower oil, babassu oil, tallow oil and rice bran oil.

Vegetable oils as referred to herein can also include processed vegetable oil material. Non-limiting examples of processed vegetable oil material include fatty acids and fatty acid alkyl esters. Alkyl esters typically include C₁-C₅ alkyl esters. One or more of methyl, ethyl, and propyl esters are preferred.

Examples of animal fats that can be used in accordance with the invention include, but are not limited to, beef fat (tallow), hog fat (lard), turkey fat, fish fat/oil, and chicken fat. The animal fats can be obtained from any suitable source including restaurants and meat production facilities.

Animal fats as referred to herein also include processed animal fat material. Non-limiting examples of processed animal fat material include fatty acids and fatty acid alkyl esters. Alkyl esters typically include C₁-C₅ alkyl esters. One or more of methyl, ethyl, and propyl esters are preferred.

Algae oils or lipids are typically contained in algae in the form of membrane components, storage products, and metabolites. Certain algal strains, particularly microalgae such as diatoms and cyanobacteria, contain proportionally high levels of lipids. Algal sources for the algae oils can contain varying amounts, e.g., from 2 wt % to 40 wt % of lipids, based on total weight of the biomass itself.

Algal sources for algae oils include, but are not limited to, unicellular and multicellular algae. Examples of such algae include a rhodophyte, chlorophyte, heterokontophyte, tribophyte, glaucophyte, chlorarachniophyte, euglenoid, haptophyte, cryptomonad, dinoflagellum, phytoplankton, and the like, and combinations thereof. In one embodiment, algae can be of the classes Chlorophyceae and/or Haptophyta. Specific species can include, but are not limited to, Neochloris oleoabundans, Scenedesmus dimorphus, Euglena gracilis, Phaeodactylum tricornutum, Pleurochrysis carterae, Prymnesium parvum, Tetraselmis chui, and Chlamydomonas reinhardtii.

Biocomponent based diesel boiling range feedstreams can typically have relatively low nitrogen and sulfur content. For example, a biocomponent based feedstream can contain up to about 300 parts per million by weight (wppm) nitrogen (in the form of nitrogen-containing compounds). Instead of nitrogen and/or sulfur, the primary heteroatom component in biocomponent based feeds is oxygen (in the form of oxygen-containing compounds). Suitable biocomponent diesel boiling range feedstreams can include up to about 10-12 wt % oxygen. In preferred embodiments, the sulfur content of the biocomponent feedstream can advantageously be about 15 wppm or less, preferably about 10 wppm or less, although, in some embodiments, the biocomponent feedstream can be substantially free of sulfur (e.g., can contain no more than 50 wppm, preferably no more than 20 wppm, for example no more than 15 wppm, no more than 10 wppm, no more than 5 wppm, no more than 3 wppm, no more than 2 wppm, no more than 1 wppm, no more than 500 wppb, no more than 200 wppb, no more than 100 wppb, no more than 50 wppb, or completely no measurable sulfur).

In some embodiments, a mineral diesel boiling range feed can be mixed with the biocomponent feed, e.g., prior to hydrodeoxygenation, preferably a relatively low value catalyst such as a water gas shift catalyst and/or an at least partially spent hydrotreating catalyst. In such embodiments, the relatively low value catalyst should preferably be selected from catalysts that have a higher relative sulfur resistance and/or the mineral feed should preferably have, or be pretreated so as to have, a relatively low sulfur and/or nitrogen content. Due to the increased sulfur content in typical mineral oil feeds, an Fe₃O₄ catalyst can rapidly be converted to some type of iron sulfide. Iron sulfides tend to have low activity for promoting hydrogenation type reactions. In such embodiments, the mineral feed can preferably be a lighter distillate feed, such as a kerosene, jet, or light gas oil feed.

When utilized, the mineral oil feedstocks can have an initial boiling point of at least about 215° F. (about 102° C.), for example at least about 250° F. (about 121° C.), at least about 275° F. (about 135° C.), at least about 300° F. (about 149° C.), at least about 325° F. (about 163° C.), or at least about 350° F. (about 177° C.). Additionally or alternately, the feedstock can be characterized by the boiling point required to boil a specified percentage of the feed. For example, the temperature required to boil at least 5 wt % of a feed is referred to as a “T5” boiling point. In one embodiment, the mineral oil feedstock can have a T5 boiling point of at least about 230° F. (about 110° C.), for example at least about 250° F. (about 121° C.) or at least about 275° F. (about 135° C.). Further additionally or alternately, the mineral hydrocarbon feed can have a T95 boiling point of about 775° F. (about 418° C.) or less, for example about 750° F. (about 399° C.) or less or about 725° F. (about 385° C.) or less.

Mineral feedstreams for blending with a biocomponent feedstream can have a nitrogen content from about 50 to about 6000 wppm nitrogen, for example from about 50 to about 2000 wppm, such as from about 75 to about 1000 wppm nitrogen. In an embodiment, feedstreams suitable for use herein can have a sulfur content from about 100 to about 40000 wppm sulfur, for example from about 200 to about 30000 wppm, such as from about 350 to about 25000 wppm. In some embodiments, the mineral stream for blending with the biocomponent stream can be a diesel boiling range stream. In other embodiments, the mineral stream can be a higher boiling range stream, such as an atmospheric or vacuum gas oil. In still other embodiments, the mineral stream can be a lower boiling range stream, such as a heavy naphtha, a virgin naphtha stream, or an other virgin distillate. Other examples of suitable mineral streams can include resid, cycle oils (e.g., light cycle oil), and coker derived oils, as well as combinations of any of these and/or any of the other aforementioned streams.

In one embodiment, the mineral stream for blending with the biocomponent stream can advantageously have relatively low nitrogen and sulfur content. In one embodiment, the mineral stream can be a finished diesel stream having a sulfur content of not more than about 50 wppm (preferably not more than about 30 wppm, for example not more than about 20 wppm, not more than about 15 wppm, not more than about 10 wppm, or not more than about 5 wppm) and a nitrogen content of not more than about 50 wppm (preferably not more than about 30 wppm, for example not more than about 20 wppm, not more than about 15 wppm, not more than about 10 wppm, or not more than about 5 wppm).

### Gas Feeds

The hydrogen source for hydrodeoxygenation of the biocomponent feedstock can be a relatively high purity hydrogen stream, e.g., containing at least about 80 mol % hydrogen, at least about 85 mol % hydrogen, at least about 90 mol % hydrogen, at least about 95 mol % hydrogen, at least about 97 mol % hydrogen, at least about 98 mol % hydrogen, or at least about 99 mol % hydrogen. Aside from hydrogen, such relatively high purity hydrogen streams may also optionally contain light side products and by-products, which can include, but are not limited to carbon oxides, light ends, water, hydrogen sulfide, ammonia, and the like, and combinations thereof. The light ends can often represent a mix of low carbon number hydrocarbons, e.g., alkanes such as methane and ethane. Additionally, as CO is a known suppressing agent for hydrotreatment reactions, increased CO content of H₂-containing gas streams can tend to reduce the overall hydrotreatment activity. Nevertheless, even though CO may at least partially suppress the activity of the catalyst, relatively high purity hydrogen treat gas compositions can contain CO in a sufficiently small amount so that the catalytic activity can preferably remain high enough to sufficiently deoxygenate the biocomponent feed (e.g., to remove the majority of the oxygen in the feed, and possibly to remove substantially all of the oxygen in the feed).

Alternately, the hydrogen source for hydroprocessing of the biocomponent feedstock can be a relatively low value refinery stream, e.g., containing 60 mol % hydrogen or less. Such refinery streams are considered of relatively low value because they have conventionally been seen as having too low of a hydrogen purity and/or too high a content of undesirable components to be used in most refinery processes. This results mostly in such refinery streams being upgraded (e.g., by separating out a higher value component for recycle and/or reuse, by separating out a lower value and/or particularly troublesome component and recycling and/or reusing the remaining components, or the like, or combinations thereof), being treated (e.g., to convert one or more lower value and/or particularly troublesome components of the refinery stream into one or more higher value components), being burned for fuel gas (typically for generating heat, e.g., to assist with temperature control of endothermic refinery reactions), and/or a combination thereof. As the desired products from certain refinery reactors are fractionated, separated, or distilled out, these light (generally gaseous) products can form a purge gas or off-gas. For example, FCC off-gas is conventionally viewed as having several problems, including having a relatively low pressure with a relatively low H₂ concentration (e.g., having a total stream pressure of about 50 psig (345 kPag), possibly up to about 100 psig (690 kPag), and a H₂ concentration of less than 25 mol %). Substantial pressure uplift would be needed for FCC off-gas to achieve hydrogen partial pressures typically indicative of hydrotreatment.

Typical refinery streams that can be useful as a hydrogen source according to this invention, aside from containing hydrogen gas, can additionally include one or more of light ends, CO, CO₂, H₂S, and NH₃. Other components may also be possible, depending on the feed chemistry and/or conditions of the reactor from which the refinery stream originated. Such a hydrogen-containing gas can be used directly as a hydrogen source for HDO of a biocomponent feed according to the invention, particularly if the hydrogen content is high enough. However, in some embodiments, the hydrogen-containing gas stream can be scrubbed prior to use, particularly when the catalyst(s) being used for the hydrodeoxygenation reaction (and/or other catalysts being used in the processes according to the invention) is(are) particularly sensitive to components present in the hydrogen-containing gas (e.g., if the gas stream contains H₂S, then it should be scrubbed prior to contacting with a sulfur-sensitive catalyst such as those comprising a Group VIII noble metals like Pt and/or Pd, whereas scrubbing of H₂S-containing gas streams would be optional when contacting a sulfur-robust catalyst such as a conventional Group VIB/VIII spent hydrotreating catalyst). Scrubbing of the hydrogen-containing gas stream can reduce (preferably significantly) the concentration of H₂S and/or CO₂ in the gas stream. An example of a suitable scrubber is an amine tower, which can use, e.g., diethylamine (DEA) or methylamine to capture H₂S and CO₂. Note that CO is typically not removed in substantial amounts by this type of scrubber.

As noted above, in certain situations, significant removal of H₂S from refinery-based hydrogen sources can be beneficial for maintaining the activity/reactivity of the catalyst(s). Preferred water gas shift catalysts according to the invention can include predominantly transition metal oxides, such as iron, chromium, copper, zinc, and combinations thereof. In the presence of H₂S, at least a portion of the catalyst may convert to sulfide(s), which can tend to be less desirable for certain catalysts such as water gas shift catalysts in the various embodiments of the invention. In some embodiments, scrubbing relatively low value hydrogen-containing refinery streams can result in a significant reduction in sulfur content, e.g., corresponding to a concentration of sulfur compounds of 15 vppm or less, for example 10 vppm or less. In certain embodiments, the scrubbed hydrogen-containing gas streams can preferably have an H₂S concentration of less than 50 vppm. These concentrations of sulfur and H₂S in scrubbed streams can advantageously be similar to the levels desired in non-refinery based and/or unscrubbed hydrogen-containing gas streams.

Upon entering a hydroprocessing (e.g., an HDO) reactor according to the present invention, the hydrogen-containing gas can preferably have a CO₂ content not to exceed 10 mol %, for example not to exceed about 7 mol %, not to exceed about 5 mol %, not to exceed about 3 mol %, not to exceed about 1 mol %, not to exceed about 5000 ppm, not to exceed about 3000 ppm, or not to exceed about 1000 ppm. In situations such as where the gas stream CO₂ content exceeds these levels, removal of CO₂ can be beneficial, e.g., for increasing the amount of hydrogen produced in situ in the reaction. The water gas shift reaction approximates an equilibrium process, where H₂O and CO can be reversibly reacted to form H₂ and CO₂, i.e., H₂O+CO⇄H₂+CO₂.

Because this resembles an equilibrium process, an increase in one of the participating species in the reaction can tend to drive the reaction to reduce that concentration. Thus, providing an excess of CO₂ can tend to drive this reaction to form more H₂O and CO. Since H₂ is a desired product from this reaction, reducing the CO₂ level should enhance the amount of H₂ produced via the water gas shift reaction.

In certain embodiments, the production of H₂ can be further enhanced by introducing steam into the reactor during a hydroprocessing step. Steam provides additional water as a reactant, which can help drive the formation of additional H₂. In some embodiments where steam is added, it may be possible to reduce the amount of steam addition once the hydrotreatment process is started, as deoxygenation of the biocomponent feed can often lead to some water production.

### Catalyst

In various embodiments, hydrodeoxygenation can be performed in the presence of a relatively low cost catalyst, such as a water gas shift catalyst and/or an at least partially spent catalyst that still has some hydrodeoxygenation activity. Water gas shift activity is typically beneficial for producing additional hydrogen (and CO₂) from H₂O (and CO) in the hydrogen source and/or in the reactor.

A preferred water gas shift catalyst includes an oxide of iron, such as Fe₃O₄. Iron oxides with water gas shift activity may be advantageous. Other water gas shift catalysts that also have hydrogenation activity can include supported catalysts such as ZnO and/or CuO, e.g., supported on alumina, iron oxide catalysts promoted with CrO₂, or the like, or combinations thereof. Water gas shift catalysts can advantageously be exposed to a reducing environment prior to use. Because biocomponent feeds typically have low sulfur contents, the catalysts should be able to maintain activity (e.g., HDO activity) for a reasonable amount of processing time (e.g., from about 6 months to about 10 years, preferably from about 1 year to about 5 years, for instance from about 18 months to about 4 years).

More generally, suitable HDO catalysts can include those hydrotreating catalysts comprising one or more Group VIII metals and one or more Group VIB metals, for example comprising Ni and/or Co and W and/or Mo, preferably comprising a combination of Ni and Mo, or Co and Mo, or a ternary combination such as Ni, Co, and Mo or such as Ni, Mo, and W. The/Each hydrotreatment catalyst can be a bulk catalyst or can be supported on an oxide such as alumina, silica, zirconia, titania, or a combination thereof, or another known support material such as carbon.

An at least partially spent NiMo catalyst can be used to initiate olefin saturation at a lower inlet temperature. Most units are constrained by a maximum operating temperature, and large amounts of heat are released from treatment of biocomponent feeds. Initiating olefin saturation at lower temperature with spent NiMo can allow for longer cycle lengths (as the maximum temperature will be reached later) and/or permits processing of more biocomponent feeds.

An at least partially spent CoMo catalyst can be used for lower hydrogen partial pressure desulfurization and to slow down the kinetics of biocomponent feed treatment. Spreading the exotherm out throughout the process by having such a lower activity catalyst can advantageously reduce the number of hotspots (which can decrease in efficiency of the unit, and potentially give rise to structural issues if near reactor walls). At high hydrogen partial pressures, the use of spent CoMo may also reduce the amount of methanation (e.g., CO+3H₂→CH₄+H₂O and/or CO₂+4H₂→CH₄+2H₂O) that occurs, which can help to reduce hydrogen consumption.

One option for the relatively low cost catalyst can be to use a spent conventional hydrotreating catalyst (e.g., NiMo and/or CoMo). Such catalyst(s) can tend to have a reduced activity for conventional hydrotreatment processes (such as HDS and/or HDN), but may still have sufficient activity for olefin saturation and/or HDO.

Another option could be to use a spent conventional hydrotreating catalyst that has been regenerated (mere reactivation of catalytically active sites) and/or rejuvenated (reactivation of catalytically active sites in combination with additional deposition of relevant metals so as to approximate the hydrotreatment activity of fresh catalyst). Though this option is possible, it is less preferred, as the regeneration and/or rejuvenation process(es) add significant expense to the process. Similarly, while fresh conventional hydrotreating catalyst, having relatively high catalytic activity in hydrotreatment, is yet another possibility, it is even less preferred than using regenerated and/or rejuvenated catalyst, as there is an additional incremental increase in cost in using such active catalysts.

As used herein, the terms “CoMo” and “NiMo” refer to catalysts comprising molybdenum and either cobalt or nickel, respectively, as catalytic metals. While sometimes fabricated as oxides, such catalysts are typically sulfided to exhibit hydrotreatment activity. Whether in oxide or sulfided form, such catalysts may also optionally include supports and minor amounts of other materials such as promoters. By way of illustration, such hydrotreating catalysts are described, for example, in one or more of U.S. Pat. Nos. 6,156,695, 6,162,350, 6,299,760, 6,582,590, 6,712,955, 6,783,663, 6,863,803, 6,929,738, 7,229,548, 7,288,182, 7,410,924, and 7,544,632, U.S. Patent Application Publication Nos. 2005/0277545, 2006/0060502, 2007/0084754, and 2008/0132407, and International Publication Nos. WO 04/007646, WO 2007/084437, WO 2007/084438, WO 2007/084439, and WO 2007/084471, inter alia. Suitable hydrotreating catalyst can also be obtained commercially, e.g., under the tradenames KF848™, KF-841™, KF840™, KF-757™, RT-601™, RT-3™, and RT-2™ from Albemarle of Baton Rouge, La.; DN-3551™, DN-3531™, DN3330™, and DN200™ from Criterion of Houston, Tex.; TK-576TH from Haldor-Topsoe of Houston, Tex.; HR626™ from Axens of Houston, Tex.; and the like.

Aside from conventional hydrodesulfurization/hydrodenitrogenation catalysts, such as CoMo and/or NiMo, the hydrodeoxygenation and hydrogenation (olefinic and/or aromatic saturation) can additionally or alternately be accomplished using fresh, regenerated and/or rejuvenated, or spent hydroisomerization/dewaxing catalyst. Such hydroisomerization/dewaxing catalysts can include molecular sieves having a SiO₂:Al₂O₃ ratio of 100 or less, for example 80 or less or 60 or less, optionally but preferably including a metal oxide binder. Non-limiting examples can include, but are not limited to, EU-1, zeolite beta, ZSM-35, ZSM-11, ZSM-57, NU-87, ZSM-22, EU-2, EU-11, ZBM-30, ZSM-48, ZSM-23, or a combination thereof, preferably zeolite beta, ZSM-48, and/or ZSM-23. In certain embodiments, the molecular sieves can have a ratio of sieve (zeolite) surface area to external surface area of at least 80:100, for example at least 90:100 or at least 105:100. When present, it can be preferable for the metal oxide binder, in powder form, to have a surface area of 100 m²/g or less, for example 80m²/g or less or 60 m²/g or less and/or to comprise at least one of silica, alumina, titania, and zirconia. Additionally or alternately, if desired, the hydroisomerizationldewaxing catalyst can also comprise a promoter metal selected from the metals of Group VIII of the Periodic Table of Elements. In embodiments where only a Group VIII metal is included, that Group VIII metal is preferably noble, more preferably comprising Pt and/or Pd. In embodiments where an additional promoter metal is provided in addition to the Group VIII metal, the Group VIII metal can be Ni and/or Co (preferably Ni), and the additional promoter metal can comprise a Group VIB metal, such as Mo and/or W, preferably at least W. By way of illustration, such hydroisomerization catalysts are described, for example, in one or more of U.S. Pat. Nos. 5,075,629, 5,110,445, 5,302,779, 5,456,820, 5,573,657, 5,723,716, 5,770,542, 5,977,425, 6,190,532, 7,077,947, 7,087,152, 7,125,818, 7,220,350, 7,282,137, 7,429,318, 7,482,300, 7,538,065, and 7,625,478, and U.S. Patent Application Publication Nos. 2005/0113250, 2006/0073961, 2008/0163672, and 2008/0171675, inter alia.

### Reaction Conditions

The feedstock (at least a portion of which is comprised of a fresh biocomponent feedstock, e.g., about 60 wt % or less, preferably about 50 wt % or less, with the remainder comprising a recycled stream and optionally a mineral feedstream), optionally steam, and hydrogen-containing gas source can be introduced into a reactor containing a catalyst having water gas shift activity, an at least partially spent hydrotreating catalyst and/or another type of catalyst having hydrodeoxygenation activity. The biocomponent-containing feed can advantageously be exposed to the catalyst under conditions effective for removing oxygen from the feed. In some embodiments, the conditions can be effective for removing substantially all of the oxygen. In alternative embodiments, the conditions can be effective for removing at least 50 wt % of the oxygen, for example at least 80 wt %, at least 85 wt %, or at least 90 wt %. In such alternative embodiments, the conditions can typically be effective for removing 99 wt % or less of the oxygen, for example 98 wt % or less or 95 wt % or less. In such alternative embodiments, the processed biocomponent feed can be mixed with a mineral diesel boiling range feed and may be co-processed in a hydrotreatment reactor to further remove oxygen from the biocomponent feed.

In situations where the biocomponent feed is exposed to the catalyst, optional steam, and hydrogen-containing gas source, the conditions can be effective for removing oxygen from the feed and/or optionally for saturating olefins. The conditions can include an LHSV from about 0.1 hr⁻¹ to about 10 hr⁻¹, for example from about 0.5 hr⁻¹ to about 1.5 hf⁻¹, and a weight average bed temperature or an estimated internal temperature (WABT or EIT, abbreviated herein as “temperature”) from about 550° F. to about 700° F. (about 288° C. to about 371° C.), for example from about 575° F. to about 675° F. (about 302° C. to about 357° C.), from about 550° F. to about 625° F. (about 288° C. to about 329° C.), from about 550° F. to about 600° F. (about 288° C. to about 315° C.), or from about 600° F. to about 650° F. (about 315° C. to about 343° C.). Note that the temperature range to can represent a balancing of hydrogenation activity for oxygen removal and water gas shift activity. Conversion of CO and H₂O into CO₂ and H₂ is an exothermic process. Because the water gas shift reaction also resembles an equilibrium process, increases in temperature can tend to drive the reaction toward CO and H₂O formation.

Based on the nature of the hydrogen source, there can be several alternatives for treat gas rate and total reaction pressure. Nevertheless, the hydrogen-containing treat gas rate for the biocomponent-containing feed can generally be at least as high as, and typically higher than, what would be used for hydrotreating a typical mineral oil feed, and the reaction pressure for the biocomponent-containing feed can advantageously be relatively low compared to what would be used for hydrotreating a typical mineral oil feed. In many embodiments, or in embodiments where refinery based streams are used as a source of hydrogen, the hydrogen in the refinery stream can optionally be supplemented with one or more other hydrogen streams of higher purity, e.g., to increase the partial pressure of hydrogen in the reactor, and thus to increase the HDO (and/or olefin saturation) activity.

In general, the total reactor pressure in the hydroprocessing reactor according to the present invention can be from about 50 psig (about 340 kPag) to about 600 psig (about 4.1 MPag), for example from about 100 psig (about 690 kPag) to about 400 psig (about 2.8 MPag), from about 50 psig (about 340 kPag) to about 300 psig (about 2.1 MPag), or from about 150 psig (about 1.0 MPag) to about 350 psig (about 2.0 MPag). Also, because there are other components besides hydrogen in the hydrogen-containing gas stream, the hydrogen partial pressure can generally be quite different from the total reactor pressure. Indeed, often in reactors containing biocomponent feeds, the hydrogen partial pressure is expressed as the partial pressure of hydrogen in the hydrogen-containing gas at the inlet to the hydroprocessing reactor; however in reactors containing only mineral feeds, the hydrogen partial pressure can generally be expressed as the to partial pressure of hydrogen at the outlet of the hydroprocessing reactor. In one embodiment, the hydrogen partial pressure (inlet) can be at least about 40 psig (about 280 kPag), for example at least about 50 psig (about 345 kPag), at least about 60 psig (about 410 kPag), at least about 75 psig (about 520 kPag), at least about 100 psig (about 690 kPag), at least about 125 psig (about 860 kPag), or at least about 150 psig (about 1.0 MPag). Additionally or alternately, the hydrogen partial pressure (inlet) can be about 500 psig (about 3.4 MPag) or less, for example about 450 psig (about 3.1 MPag) or less, about 400 psig (about 2.8 MPag) or less, about 350 psig (about 2.4 MPag) or less, about 300 psig (about 2.1 MPag) or less, about 250 psig (about 1.7 MPag) or less, about 200 psig (about 1.4 MPag) or less, or about 150 psig (about 1.0 MPag) or less.

Depending on the size of the relevant reactor, the amount of biocomponent feed processed using the hydrogen-containing gas stream can be adjusted to yield a desired treat gas ratio of hydrogen source to biocomponent feed. Nevertheless, the treat gas ratio for the hydrodeoxygenation reaction can generally be from about 500 scf/bbl to about 5000 scf/bbl (about 85 Nm³/m³ to about 850 Nm³/m³), for example from about 750 scf/bbl to about 3000 scf/bbl (about 130 Nm³/m³ to about 510 Nm³/m³), from about 750 scf/bbl to about 2500 scf/bbl (about 130 Nm³/m³ to about 470 Nm³/m³), from about 900 scf/bbl to about 2500 scf/bbl (about 150 Nm³/m³ to about 470 Nm³/m³), or from about 1000 scf/bbl to about 3000 scf/bbl (about 170 Nm³/m³ to about 510 Nm³/m³).

In some embodiments, some portions of the reaction can be performed in a counter-current reactor. In such a situation, the direction of flow for the biocomponent feed during processing would be opposite from the direction of flow for the hydrogen-containing gas stream. When present, steam may also typically flow in the opposite direction from the biocomponent feed during counter-current operation. In embodiments including a counter-current reactor, the feed can enter the reactor at the top, while the hydrogen-containing gas and steam flows can enter at the bottom. As the gas travels up the reactor, it can typically become depleted of hydrogen, generally leading to lower hydrogen partial pressures near the top (outlet) of the reactor. One advantage of this counter-current scheme can be that the lowest hydrogen partial pressures can encounter the feed when it first enters the reactor. The lower partial pressure of hydrogen can be sufficient to saturate olefins within the biocomponent feed, leaving the higher hydrogen pressure at the bottom of the reactor to effectuate HDO.

Another potential advantage of a counter-current design can be the ability to modify the temperature profile across the reactor, e.g., to enhance hydrogen production via water gas shift. In the water gas shift reaction, production of hydrogen (and CO₂) from water and CO is exothermic. Thus, decreasing the temperature in the reaction environment can cause the water gas shift equilibrium to favor more hydrogen production. In an embodiment, the temperature can be varied within the counter-current reactor so that lower reaction temperatures are found near the top of the reactor and so that higher reaction temperatures are found near the bottom.

The reaction can produce up to three types of effluent streams (or more). One type of stream can include a vapor effluent stream. The vapor effluent can include unreacted H₂, any CO and/or CO₂, water vapor, light ends, and any other light products, e.g., that may have been introduced to the reactor with the hydrogen-containing gas stream. The vapor effluent can be used as a fuel gas, when the light ends content of this stream is high enough to have fuel value. Another type of effluent stream can include a diesel boiling range product stream, which can advantageously comprise a majority of the processed biocomponent feedstock. The third type of stream can include an aqueous output stream, which can comprise water introduced with the biocomponent feed and/or from steam used to drive the water gas shift reaction. This aqueous stream can also include some level of dissolved organics and other particulate and/or dissolved impurities, which can, in some embodiments, be passed to a waste treatment facility.

In one embodiment, the fresh biocomponent portion of the feedstock can be pretreated to remove impurities prior to hydrotreatment. When desired, this pretreatment can, in some embodiments, occur prior to mixing the biocomponent portion of the feedstock with the mineral oil portion. In such embodiments, the pretreatment can include passing the biocomponent portion through an adsorbent to remove metals, filtering the biocomponent portion to remove sediment, or other processes. Alternately, an optional metals removal pretreatment can take place after mixing of the biocomponent and mineral oil feeds, by exposing the combined feedstock to a demetallization catalyst under demetallization conditions prior to hydrodeoxygenation (and optionally hydrodesulfurization).

In other embodiments, at least a portion (e.g., about 50 wt % or less) of the biocomponent portion of the feedstock can advantageously be fresh (i.e., not pretreated). In such embodiments, the remainder of the feedstock can be comprised of recycled hydrodeoxygenated biocomponent feed, hydrotreated mineral feed (e.g., having a diesel boiling range, an atmospheric gasoil boiling range, and/or a vacuum gasoil boiling range), and/or fresh mineral feed (e.g., in a kerosene boiling range) having an appropriately low sulfur and nitrogen content.

In some embodiments, at least a portion of the diesel boiling range (hydrodeoxygenated) product stream can be suitable for incorporation into the diesel fuel pool without further processing. Additionally or alternately, at least a portion of the diesel boiling range product stream can be subjected to further processing. One type of further processing can include removal of at least a portion of any undesirable heteroatoms (e.g., nitrogen and/or sulfur) remaining in the diesel boiling range product. In the case of sulfur, this can be done using a caustic solution or other wash to remove hydrogen sulfides, or through a sulfur adsorption step, such as by exposing the liquid stream to metallic (massive) Ni, ZnO, or another adsorber of sulfur species, in order to remove mercaptans and/or carbonyl sulfides. Another type of further processing can include recycling of a portion of the product to the inlet of the reactor, e.g., for temperature control. Still another alternative can include stripping the diesel boiling range product of dissolved gases. In some embodiments, two or more further processing steps can be undertaken, e.g., recycling and stripping.

Additional hydroprocessing of the diesel boiling range (hydrodeoxygenated) product is also possible. In some embodiments, the diesel boiling range (hydrodeoxygenated) product can be exposed to a hydroisomerization catalyst under effective hydroisomerization conditions. Performing a hydroisomerization process on the diesel boiling range product can improve the cold-flow properties of the product. Advantageously, the hydroisomerization reaction could also simultaneously remove low levels of sulfur and/or oxygen from the diesel boiling range product. In some embodiments, this hydroisomerization can be conducted on a portion of the diesel boiling range (hydrodeoxygenated) product to be recycled.

In the optional hydroisomerization stage, the diesel boiling range product can be exposed to one or more reaction zones, optionally present in a separate reactor, that are operated at hydroisomerization conditions in the presence of hydroisomerization catalyst. Generally, catalytic dewaxing can be accomplished by selective hydrocracking or by hydroisomerizing long chain molecules within a feed such as a diesel boiling range feed. Dewaxing catalysts can include, but are not necessarily limited to, molecular sieves such as crystalline aluminosilicates (zeolites) or silicoaluminophosphates (SAPOs). These molecular sieve catalysts may also carry a metal hydrogenation component, such as a Group VIII metal (such as Ni and/or Co, in some preferred embodiments of which the metal hydrogenation component can additionally include a Group VIB metal such as Mo and/or W), in some cases a Group VIII noble metal (such as Pt and/or Pd). Conditions for hydroisomerization/dewaxing can include temperatures from about 250° C. to about 450° C., for example from about 280° C. to about 380° C., total pressures from about 100 psig to about 3000 psig (about 690 kPag to about 20.7 MPag), LHSV values from about 0.1 hr⁻¹ to about 10 hr⁻¹, and treat gas ratios from about 500 scf/bbl to about 5000 scf/bbl (about 85 Nm³/m³ to about 850 Nm³/m³).

In various embodiments, the molecular sieve used for catalytic hydroisomerization/dewaxing can comprise an aluminosilicate, e.g., having an MRE framework zeolite such as ZSM-48, which is a 10-membered ring molecular sieve having a 1-D channel structure. ZSM-48-type molecular sieves can perform dewaxing primarily by isomerizing molecules within the feed. Typical silica to alumina ratios for the aluminosilicate can be from about 250:1 or less, or from 200:1. Preferably, the silica to alumina ratio of the aluminosilicate can be less than about 110:1, for example less than about 110:1 to about 20:1 or from about 100:1 to about 40:1. To form a catalyst, the molecular sieve can be composited with a binder. Suitable binders can include, but are not limited to silica, alumina, silica-alumina, titania, zirconia, or a mixture thereof. Other suitable binders will be apparent to those of skill in the art.

In a particularly advantageous embodiment, the optional hydroisomerization/dewaxing treatment can improve in the hydroisomerized/dewaxed product one or more of the following: sulfur content (i.e., by lowering it); cetane number; and one or more cold flow properties (such as pour point, cloud point, low-temperature viscosity, and the like).

Another, less preferred hydroprocessing option can include mixing the diesel boiling range product with a mineral diesel feed and then hydrotreating the mixed feed. This option can be preferred, particularly in cases where initial hydrotreating (e.g., HDO) of the biocomponent feed does not sufficiently lower the heteroatom (e.g., oxygen) content. By removing a majority of the heteroatoms (e.g., oxygen) in the initial hydrotreating (e.g., HDO) stage, suppression of catalytic activity due to the remaining heteroatoms (e.g., oxygen) can be reduced or minimized. Thus, a subsequent hydrotreatment for the mixed biocomponent and mineral feed can advantageously remove additional oxygen and/or can remove additional sulfur and/or nitrogen for the mineral feed. Subsequent hydrotreatment of the mixed biocomponent and mineral feed can advantageously also produce a diesel boiling range product. This diesel boiling range product can be added to the diesel fuel pool, or it can undergo any of the types of further processing mentioned above.

In embodiments where a mineral feed is mixed with the biocomponent feed prior to a hydrotreatment step, the mineral feed can preferably be a diesel boiling range feed. Untreated mineral feedstreams for blending with a biocomponent feedstream can have a nitrogen content from about 50 to about 6000 wppm nitrogen, for example from about 50 to about 2000 wppm, such as from about 75 to about 1000 wppm nitrogen. In an embodiment, untreated mineral feedstreams can have a sulfur content from about 100 to about 40000 wppm sulfur, for example from about 200 to about 30000 wppm, such as from about 350 to about 25000 wppm. Suitable diesel boiling range feedstreams can boil within the range of about 215° F. (about 102° C.) to about 800° F. (about 427° C.). Preferably, the diesel boiling range feedstream has an initial boiling point of at least about 250° F. (about 121° C.), or at least about 300° F. (about 149° C.), or at least about 350° F. (about 177° C.), or at least about 400° F. (about 204° C.), or at least about 451° F. (about 233° C.). Preferably, the diesel boiling range feedstream has a final boiling point of about 800° F. (about 427° C.) or less, for example about 775° F. (about 413° F.) or less or about 750° F. (about 399° C.) or less. In an embodiment, the diesel boiling range feedstream has a boiling range from about 451° F. (233° C.) to about 800° F. (427° C.). In another embodiment, the diesel boiling range feedstream can also include kerosene range compounds to provide a feedstream with a boiling range from about 250° F. (121° C.) to about 800° F. (427° C.).

In embodiments where a hydrotreated mineral feed comprises at least a portion of the remainder of the hydrodeoxygenation reaction feedstock, the mineral feed can be exposed to a hydrogen source in the presence of a hydrotreating catalyst under hydrotreating conditions, in order to reduce the levels of sulfur and/or nitrogen therein to a desirable level, e.g., where they may not substantially hinder the subsequent hydrodeoxygenation reaction. Such hydrotreating conditions can include, but are not limited to, one or more of an LHSV from about 0.1 hr⁻¹ to about 10 hr⁻¹, a total pressure from about 250 psig to about 2000 psig (about 1.7 MPag to about 13.8 MPag), a temperature from about 550° F. to about 750° F. (about 288° C. to about 399° C.), and a hydrogen treat gas rate from about 750 scf/bbl to about 3000 scf/bbl (about 130 Nm³/m³ to about 510 Nm³/m³) of at least about 80 mol % hydrogen.

The sources of hydrogen for the hydrodeoxygenation reactor and for any other reactors/stages requiring hydrogen, such as for hydroisomerization and/or for hydrotreatment, when present, can come from the same source, e.g., a refinery hydrogen network, or can come from different sources. Nevertheless, it is preferable that all hydrogen sources according to the invention are of relatively high hydrogen purity, although it is contemplated that some or all of such hydrogen sources can be less pure, in which case the reaction conditions can be altered to compensate for the lower hydrogen content in the treat gas and/or additional hydrogen can be added into the reactor(s) to compensate.

### Reaction System

The reactor used for hydrodeoxygenating the biocomponent feed can be a hydroprocessing reactor, such as a reactor suitable for performing a hydrotreatment process. The reactor can be configured to operate in a co-current or counter-current manner. If the reactor is configured for counter-current operation, the reactor can be a fixed bed reactor in one embodiment, with the biocomponent feed flowing in the opposite direction relative to both the hydrogen-containing gas flow and, if present, the steam flow. If the reactor is configured for co-current operation, either fixed or fluidized beds may be used.

The reaction system can include several inputs for the reactor. In addition to the biocomponent feedstock, input conduits can also be available for the hydrogen-containing gas and optionally for steam. The input conduit for the hydrogen-containing gas can receive the gas stream from an optional scrubber that can remove at least a portion of the H₂S and at least a portion of the CO₂ present in the gas stream. The reaction system can also include a catalyst. If the reactor is a fixed bed reactor, the reactor may include one or multiple beds of catalyst. The effluent from the reactor can be sent to a separator, e.g., to produce three output streams, including a vapor effluent, an aqueous effluent, and a diesel boiling range product.

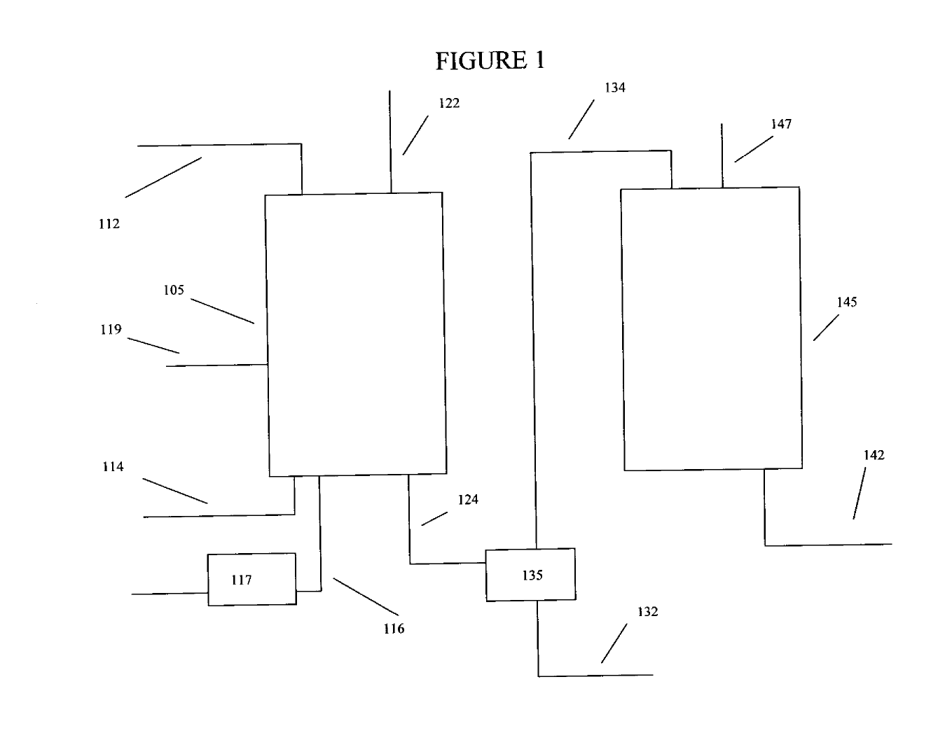

A reaction system suitable for performing an embodiment of the invention is schematically shown in FIG. 1. In FIG. 1, reactor 105 has several input conduits. Feed conduit 112 provides a biocomponent feedstock for processing. Steam conduit 114 (optional) can provide steam to reactor 105. Conduit 116 provides to the reactor a hydrogen source comprising a relatively low hydrogen content gas. As needed or desired, the hydrogen-containing gas can pass through scrubber 117 (optional) prior to entering conduit 116 for passage to reactor 105. Based on the arrangement of input feeds, reactor 105 is configured for counter-current operation; however, it will be appreciated that co-current reactor operation can be effected with appropriate rearrangement of the layout in FIG. 1. Optionally, an additional hydrogen feed 119 may be provided to the reactor, e.g., to increase the hydrogen partial pressure as necessary. As shown in FIG. 1, the additional hydrogen feed 119 may be added at an intermediate point in the reactor. However, additional hydrogen can additionally or alternately be introduced through or more proximal to conduit 116 (and/or may be passed through optional scrubber 117), along with other hydrogen and/or other gases.

Products from reactor 105 can exit via one or more conduits. Vapor conduit 122 provides an exit for gases and light products, e.g., that can be sent to a different unit for purification and/or recycled to another portion of the refinery or that can used as fuel gas. Liquid conduit 124 provides an exit for liquid effluent and links to a separator 135, where an aqueous effluent 132 can be separated from a diesel boiling range product 134. Separator 135 can also separate out any remaining gases and/or light products from diesel boiling range product 134. As shown in FIG. 1, diesel boiling range product 134 can be passed into optional hydroisomerization reactor 145. Therein, the diesel boiling range product can be exposed to a hydroisomerization catalyst in the presence of hydrogen from hydrogen input 147 to produce hydroisomerized diesel boiling range product 142.

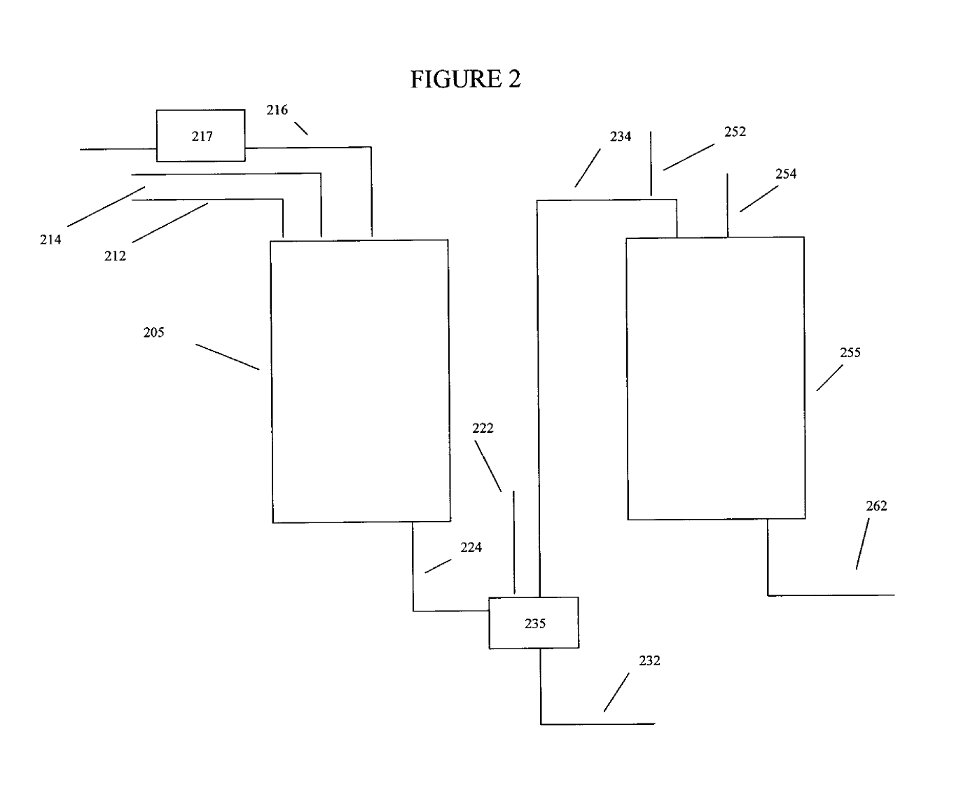

Another reaction system suitable for performing an embodiment of the invention is schematically shown in FIG. 2. In FIG. 2, reactor 205 has several input conduits. Feed conduit 212 provides a biocomponent feedstock for processing. Steam conduit 214 (optional) can provide steam to reactor 205. Conduit 216 provides to the reactor a hydrogen source comprising a relatively low hydrogen content gas. As needed or desired, the hydrogen-containing gas can pass through scrubber 217 (optional) prior to entering conduit 216 for passage to reactor 205. Based on the arrangement of input feeds, reactor 205 is configured for co-current operation; however, it will be appreciated that counter-current reactor operation can be effected with appropriate rearrangement of the layout in FIG. 2.

Effluent 224 from reactor 205 can enter separator 235, which can separate out vapor phase product 222 (e.g., that can be sent to a different unit for purification and/or recycled to another portion of the refinery or that can used as fuel gas), an aqueous effluent 232 for waste or further treatment (not shown), and diesel boiling range product 234. As shown in FIG. 2, diesel boiling range product 234 can optionally be mixed with a mineral diesel boiling range feed 252 and can then be passed into second hydrotreatment reactor 255, optionally along with additional hydrogen 254. The mixed feed can be hydrotreated in reactor 255 to produce mixed diesel boiling range product 262. This mixed diesel boiling range product can optionally be passed through a second separator (not shown) to remove contaminant gases, such as H₂S, prior to being used directly or added to the diesel fuel pool.

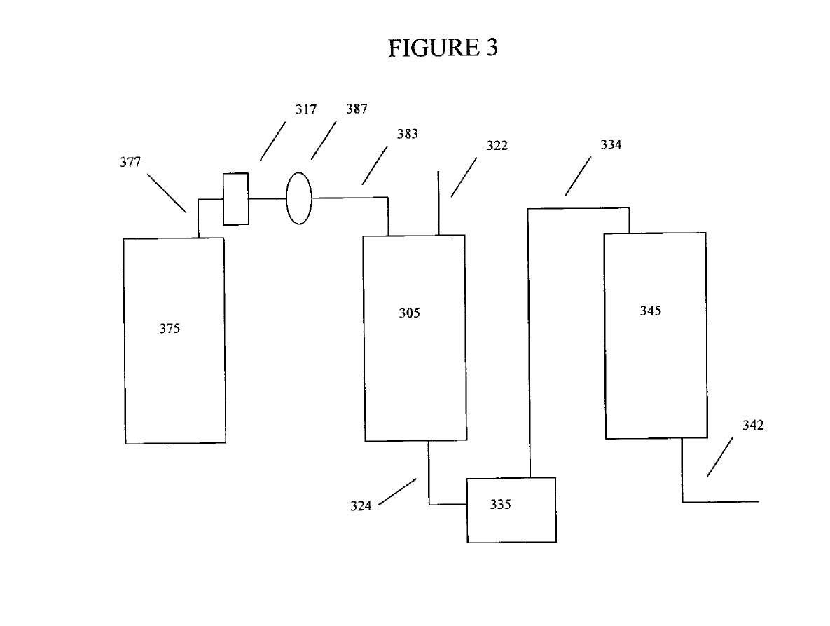

FIG. 3 schematically shows the overall integration of a reaction system according to an embodiment of the invention within a refinery. In FIG. 3, the connectivity is shown between a refinery reactor 375 and hydroprocessing reactor 305 for processing a biocomponent feed. Refinery reactor 375, having hydrogen-containing gas exit conduit 377 can represent any refinery reactor from which can emanate a stream containing from about 20 mol % to about 60 mol % hydrogen. The hydrogen-containing gas can pass through conduit 377, optionally through scrubber 317, and into intervening conduit 383. In FIG. 3, an optional pump 387 is also shown, if desired for increasing the pressure of the hydrogen-containing gas flow. Intervening conduit 383 introduces the optionally scrubbed and/or pressurized hydrogen-containing gas flow to hydroprocessing reactor 305. Hydroprocessing reactor 305 also receives biocomponent feed 322, and optionally also steam (not shown). After hydroprocessing, the liquid effluent can be passed to separator 335 via liquid effluent conduit 324. In some embodiments, liquid effluent conduit 324 can also include a vapor effluent, and separator 335 can also separate out this vapor, if present. Separator 335 can also produce a diesel boiling range product 334, which can optionally be passed to another hydroprocessing reactor, such as a hydroisomerization reactor 345. In such situation, the hydroisomerized diesel boiling range product 342 can be used directly or delivered to the diesel fuel pool.

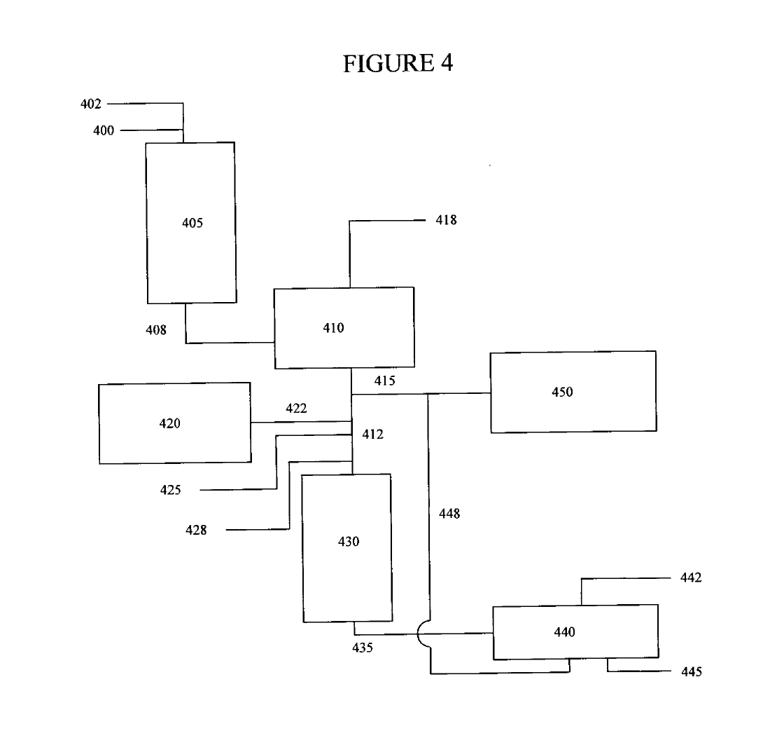

FIG. 4 schematically shows an embodiment in which a pre-hydrotreated mineral oil feedstock is mixed with a biocomponent feedstock before undergoing hydrodeoxygenation with a reduced hydrogen content gas and/or a reduced activity catalyst. In FIG. 4, mineral oil feedstock 400 can be fed to hydrotreatment reactor 405, along with hydrogen-containing treat gas 402. While this reactor 405 can be a conventional hydrotreatment reactor, typically using a relatively high activity catalyst for performing hydrodenitrogenation and hydrodesulfurization and typically using conventional treat gas containing a relatively high hydrogen content (i.e., at least 80 mol % or vol % hydrogen, and usually at least 90 mol % or vol % hydrogen, generally with the remainder being relatively inert gas), it can be possible for the catalyst activity to be lower than conventional and/or for the hydrogen content to be lower than conventional. Hydrotreated mineral oil effluent can exit reactor 405 through conduit 408, which leads to separator 410, which may include a single separator or a series of separators, such as relatively higher temperature and relatively lower temperature separators.

It should be appreciated that there may be other inputs to and/or exits from reactor 405, which are not shown in FIG. 4, e.g., an exit for hydrotreatment vapor phase effluent, which can optionally be recycled as a hydrogen-containing gas to reactor 430 and/or can be used in other refinery operations, e.g., as fuel gas. Separator 410 can yield a (liquid phase) hydrotreated mineral oil stream 412 and a vapor phase effluent 418, the hydrogen within which can advantageously be recycled to the treat gas loop, e.g., whence treat gas 402 can originate. In a refinery that processes only mineral oil feeds, hydrotreated mineral oil stream 412 would be directly diverted through conduit 415 into the ADO pool 450. The ADO pool 450 may include a stripper (not shown), if desired. Also, if hydrotreated mineral oil 415 was desired to have further treatment, such as further hydrotreatment, dewaxing, or the like (not shown), that can additionally or alternately be undertaken before proceeding to ADO pool 450.

However, in embodiments where mineral oil and biocomponent feedstocks are to be co-processed, all or a portion of hydrotreated mineral oil stream 412 can pass instead to hydrodeoxygenation reactor 430. The biocomponent feedstock 422 from tank 420 (which can represent a storage tank or an unnamed source of biocomponent feedstock) can be mixed with hydrotreated mineral oil stream 412. Also, hydrogen-containing gas 425, preferably containing to relatively low hydrogen content, such as from about 20 mol % to about 60 mol % hydrogen, e.g., from a refinery stream purge gas or off-gas, can be added. Other optional additions can be made through line 428, e.g., steam, additional hydrogen, mineral oil feed that had not been pre-hydrotreated, or the like, or combinations thereof. In FIG. 4, these additions to the hydrotreated mineral oil stream 412 are shown as occurring prior to entering the reactor 430, but it is envisioned that this mixing could additionally or alternately occur within the reactor 430. Similarly to reactor 405, it should be appreciated that there may be other inputs to and/or exits from hydrotreatment reactor 430, which are not shown in FIG. 4, e.g., an exit for hydrotreatment vapor phase effluent, which can optionally be recycled as a hydrogen-containing gas to reactor 430 and/or can be used in other refinery operations, e.g., as fuel gas.

The (liquid phase) hydrotreated mixed mineral and biocomponent effluent 435 from reactor 430, like stream 408, can be further processed using separator 440. Separator 440 can yield a (liquid phase) hydrotreated renewable diesel boiling stream 448, a vapor phase effluent 442, and optionally a sour water stream 445. If the hydrogen content in vapor phase effluent 442 is high enough, the effluent 442 can be recycled as a hydrogen-containing gas to reactor 430, and/or the effluent 442 can be used in other refinery operations, e.g., as fuel gas. Sour water stream 445, when present, can be sent for further processing or to waste (not shown). Renewable diesel boiling stream 448 can advantageously be sent to ADO pool 450, optionally being combined with any portion of the pre-hydrotreated mineral oil stream 415 not mixed with biocomponent feedstock via 422. Also, if renewable diesel boiling stream 448 was desired to have further treatment, such as further hydrotreatment, dewaxing, or the like (not shown), that can additionally or alternately be undertaken before proceeding to ADO pool 450.

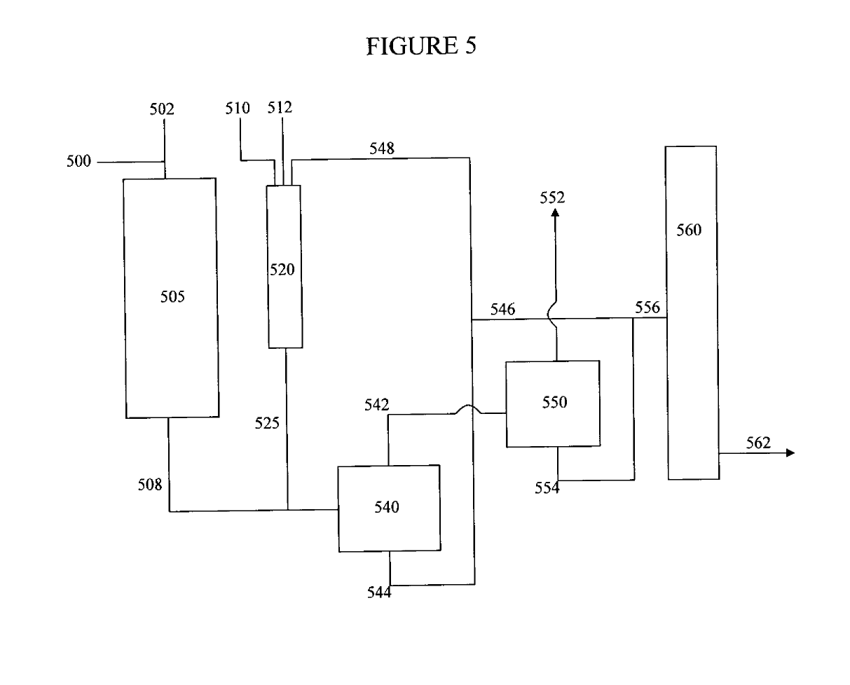

FIG. 5 schematically shows an embodiment in which a mineral oil feedstock can be hydrotreated in parallel with a biocomponent feedstock being hydrodeoxygenated with in the presence of a reduced activity catalyst. In FIG. 5, hydrodeoxygenation reactor 520 contains a hydrodeoxygenation catalyst (e.g., a water gas shift catalyst and/or an at least partially spent catalyst having a hydrodeoxygenation activity, such as spent hydrotreating catalyst) and has several input conduits. Feed conduit 510 provides a fresh biocomponent feedstock for processing. Steam conduit (not shown, optional) can provide steam to reactor 520, if desired. Conduit 512 provides to the reactor a hydrogen source comprising a relatively pure hydrogen content gas (e.g., at least about 80 mol % hydrogen, such as at least 95 mol % hydrogen). Based on the arrangement of input feeds, reactor 520 is configured for co-current operation; however, it will be appreciated that counter-current reactor operation can be effected with appropriate rearrangement of the inputs, such as into reactor 105 of FIG. 1. Hydrodeoxygenated product from reactor 520 can exit via conduit 525.

As shown in FIG. 5, in parallel, mineral feedstock 500 can enter hydrotreatment reactor 505 for catalytic hydroprocessing using a hydrotreating catalyst (e.g., a bulk and/or supported catalyst containing Group VIII/Group VIB metals and typically sulfided, such as a NiMo, a CoMo, and/or a NiMoW catalyst). Steam conduit (not shown, optional) can provide steam to reactor 505, if desired. Conduit 502 provides to the reactor a hydrogen source, generally comprising a relatively pure hydrogen content gas (e.g., at least about 80 mol % hydrogen, such as at least 95 mol % hydrogen). Based on the arrangement of input feeds, reactor 505 is configured for co-current operation; however, it will be appreciated that counter-current reactor operation can be effected with appropriate rearrangement of the inputs, such as into reactor 105 of FIG. 1. Hydrotreated product from reactor 505 can exit via conduit 508.

While it should be appreciated by one of ordinary skill in the art that the hydrotreated product in conduit 508 and the hydrodeoxygenated product in conduit 525 can individually undergo separation, FIG. 5 shows that these streams can be merged for combined separation in hot separator drum 540 into a hot vapor phase component that can exit through conduit 542, optionally a hot aqueous phase (not shown), and a hot liquid phase component that can exit through conduit 544. At least a portion of the separated hot liquid phase component can advantageously be recycled to the hydrodeoxygenation reactor 520 through conduit 548, e.g., in order to be further hydrodeoxygenated and/or in order to act as a diluent to manage the large exotherms common in biocomponent hydrodeoxygenation reactions. Optionally but preferably, another portion of the hot separated liquid phase component, which has sufficiently low oxygen, nitrogen, and sulfur content, can be sent to the fuel pool, e.g., as diesel fuel, through conduit 562. In FIG. 5, however, this other portion of the separated hot liquid phase component may not have sufficiently low heteroatom levels, in which case it is optionally but preferably first sent through conduit 546 to stripper 560 before being directed to the fuel pool through conduit 562.

While it is shown in FIG. 5 that hydrogen sources 502 and 512 for reactors 505 and 520, respectively, are independent, they can instead be drawn from the same source, e.g., a refinery distribution network hydrogen source.

FIG. 5 shows the vapor phase component that exits hot separator drum 540 through conduit 542 being directed to a cold separator drum 550, which itself can create a cold vapor phase component that can exit through conduit 552, optionally a cold aqueous phase (not shown), and a cold liquid phase component that can exit through conduit 554. A portion (though none in FIG. 5) of the cold separated liquid phase component, which has sufficiently low oxygen, nitrogen, and sulfur content, can be sent to the fuel pool, e.g., as diesel fuel, through conduit 562. As with the hot separated liquid phase component, in FIG. 5, the cold separated liquid phase component may not have sufficiently low heteroatom levels, in which case it is optionally but preferably first sent through conduit 556 (where it is combined with the hot separated liquid phase component from conduit 546) to stripper 560 before being directed to the fuel pool through conduit 562.

The cold vapor phase component in conduit 552 can be: sent to flare; used as fuel gas; purified to isolate one or more higher value components (such as hydrogen) therefrom; recycled and/or cascaded to the hydrodeoxygenation reactor 520, the hydrotreatment reactor 505, and/or another refinery process needing hydrogen or another gaseous component; or the like; or some combination thereof. Although FIG. 5 shows a two-stage separation using hot separator drum 540 and cold separator drum 550, it is of course contemplated that more than two separation stages may be used, or alternately that only one separation stage may be used, in which latter case the vapor phase component 542 from sole separator drum 540 can be disposed of as detailed for cold vapor phase component in conduit 552 above, with separator 550 and conduits 552 and 554 being removed from FIG. 5.

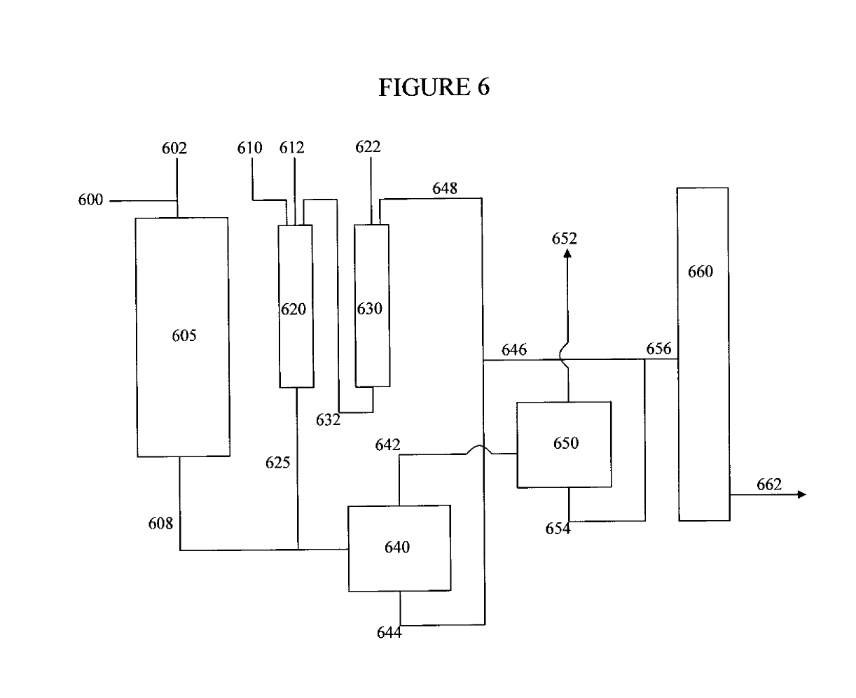

FIG. 6 schematically shows an embodiment in which a mineral oil feedstock can be hydrotreated in parallel with a biocomponent feedstock being hydrodeoxygenated with in the presence of a reduced activity catalyst. In FIG. 6, hydrodeoxygenation reactor 620 contains a hydrodeoxygenation catalyst (e.g., a water gas shift catalyst and/or an at least partially spent catalyst having a hydrodeoxygenation activity, such as spent hydrotreating catalyst) and has several input conduits. Feed conduit 610 provides a fresh biocomponent feedstock for processing. Steam conduit (not shown, optional) can provide steam to reactor 620, if desired. Conduit 612 provides to the reactor a hydrogen source comprising a relatively pure hydrogen content gas (e.g., at least about 80 mol % hydrogen, such as at least 95 mol % hydrogen). Recycle conduit 632 can also provide an additional hydrocarbon feed. Based on the arrangement of input feeds, reactor 620 is configured for co-current operation; however, it will be appreciated that counter-current reactor operation can be effected with appropriate rearrangement of the inputs, such as into reactor 105 of FIG. 1. Hydrodeoxygenated product from reactor 620 can exit via conduit 625.

As shown in FIG. 6, in parallel, mineral feedstock 600 can enter hydrotreatment reactor 605 for catalytic hydroprocessing using a hydrotreating to catalyst (e.g., a bulk and/or supported catalyst containing Group VIII/Group VIB metals and typically sulfided, such as a NiMo, a CoMo, and/or a NiMoW catalyst). Steam conduit (not shown, optional) can provide steam to reactor 605, if desired. Conduit 602 provides to the reactor a hydrogen source, generally comprising a relatively pure hydrogen content gas (e.g., at least about 80 mol % hydrogen, such as at least 95 mol % hydrogen). Based on the arrangement of input feeds, reactor 605 is configured for co-current operation; however, it will be appreciated that counter-current reactor operation can be effected with appropriate rearrangement of the inputs, such as into reactor 105 of FIG. 1. Hydrotreated product from reactor 605 can exit via conduit 608.

While it should be appreciated by one of ordinary skill in the art that the hydrotreated product in conduit 608 and the hydrodeoxygenated product in conduit 625 can individually undergo separation, FIG. 6 shows that these streams can be merged for combined separation in hot separator drum 640 into a hot vapor phase component that can exit through conduit 642, optionally a hot aqueous phase (not shown), and a hot liquid phase component that can exit through conduit 644. At least a portion of the separated hot liquid phase component can advantageously be recycled through conduit 648. However, unlike in FIG. 5, this recycle first goes through a hydroisomerization reactor 630, which contains a hydroisomerization catalyst (e.g., a zeolite-supported catalyst containing a noble Group VIII metal or a non-noble Group VIII metal and a Group VIB metal, generally sulfided, such as a Pt- and/or Pd-promoted zeolite Y dewaxing catalyst, a Pt-promoted ZSM-48 dewaxing catalyst, and/or a NiW-promoted ZSM-48 dewaxing catalyst) and is fed with a hydrogen source 622. Recycling the dewaxed/isomerized product through conduit 632 can allow a feed to be further hydrodeoxygenated, to have advantageously better cold-flow properties (e.g., lower relative cloud point, lower relative pour point, lower relative cold filter plugging point, or the like, or a combination thereof) for managing the tendency of hydrodeoxygenated biocomponent products to have relatively poor (e.g., off-spec high) cold-flow properties, and/or to act as a diluent to manage the large exotherms common in biocomponent hydrodeoxygenation reactions.

Optionally but preferably, another portion of the hot separated liquid phase component, which has sufficiently low oxygen, nitrogen, and sulfur content, can be sent to the fuel pool, e.g., as diesel fuel, through conduit 662. In FIG. 6, however, this other portion of the separated hot liquid phase component may not have sufficiently low heteroatom levels, in which case it is optionally but preferably first sent through conduit 646 to stripper 660 before being directed to the fuel pool through conduit 662. While it is shown in FIG. 6 that hydrogen sources 602, 612, and 622 for reactors 605, 620, and 630, respectively, are independent, they can instead be drawn from the same source, e.g., a refinery distribution network hydrogen source.

FIG. 6 shows the vapor phase component that exits hot separator drum 640 through conduit 642 being directed to a cold separator drum 650, which itself can create a cold vapor phase component that can exit through conduit 652, optionally a cold aqueous phase (not shown), and a cold liquid phase component that can exit through conduit 654. A portion (though none in FIG. 6) of the cold separated liquid phase component, which has sufficiently low oxygen, nitrogen, and sulfur content, can be sent to the fuel pool, e.g., as diesel fuel, through conduit 662. As with the hot separated liquid phase component, in FIG. 6, the cold separated liquid phase component may not have sufficiently low heteroatom levels, in which case it is optionally but preferably first sent through conduit 656 (where it is combined with the hot separated liquid phase component from conduit 646) to stripper 660 before being directed to the fuel pool through conduit 662.

The cold vapor phase component in conduit 652 can be: sent to flare; used as fuel gas; purified to isolate one or more higher value components (such as hydrogen) therefrom; recycled and/or cascaded to the hydrodeoxygenation reactor 620, the hydrotreatment reactor 605, the hydroisomerization reactor 630, and/or another refinery process needing hydrogen or another gaseous component; or the like; or some combination thereof. Although FIG. 6 shows a two-stage separation using hot separator drum 640 and cold separator drum 650, it is of course contemplated that more than two separation stages may be used, or alternately that only one separation stage may be used, in which latter case the vapor phase component 642 from sole separator drum 640 can be disposed of as detailed for cold vapor phase component in conduit 652 above, with separator 650 and conduits 652 and 654 being removed from FIG. 6.

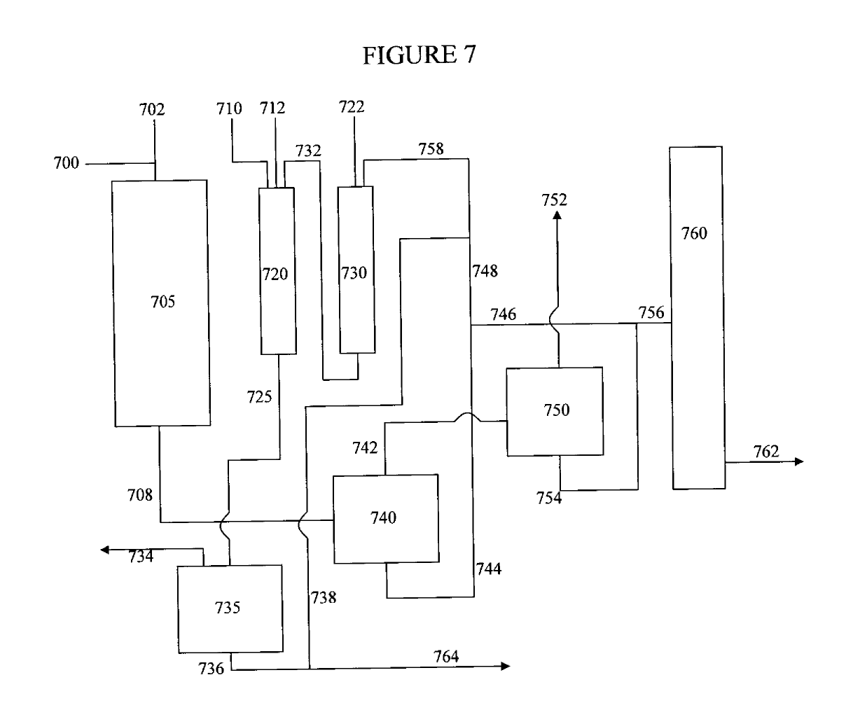

FIG. 7 schematically shows an embodiment in which a mineral oil feedstock can be hydrotreated in parallel with a biocomponent feedstock being hydrodeoxygenated with in the presence of a reduced activity catalyst. In FIG. 7, hydrodeoxygenation reactor 720 contains a hydrodeoxygenation catalyst (e.g., a water gas shift catalyst and/or an at least partially spent catalyst having a hydrodeoxygenation activity, such as spent hydrotreating catalyst) and has several input conduits. Feed conduit 710 provides a fresh biocomponent feedstock for processing. Steam conduit (not shown, optional) can provide steam to reactor 720, if desired. Conduit 712 provides to the reactor a hydrogen source comprising a relatively pure hydrogen content gas (e.g., at least about 80 mol % hydrogen, such as at least 95 mol % hydrogen). Recycle conduit 732 can also provide an additional hydrocarbon feed. Based on the arrangement of input feeds, reactor 720 is configured for co-current operation; however, it will be appreciated that counter-current reactor operation can be effected with appropriate rearrangement of the inputs, such as into reactor 105 of FIG. 1. Hydrodeoxygenated product from reactor 720 can exit via conduit 725.

As shown in FIG. 7, in parallel, mineral feedstock 700 can enter hydrotreatment reactor 705 for catalytic hydroprocessing using a hydrotreating catalyst (e.g., a bulk and/or supported catalyst containing Group VIII/Group VIB metals and typically sulfided, such as a NiMo, a CoMo, and/or a NiMoW catalyst). Steam conduit (not shown, optional) can provide steam to reactor 705, if desired. Conduit 702 provides to the reactor a hydrogen source, generally comprising a relatively pure hydrogen content gas (e.g., at least about 80 mol % hydrogen, such as at least 95 mol % hydrogen). Based on the arrangement of input feeds, reactor 705 is configured for co-current operation; however, it will be appreciated that counter-current reactor operation can be effected with appropriate rearrangement of the inputs, such as into reactor 105 of FIG. 1. Hydrotreated product from reactor 705 can exit via conduit 708.

While it should be appreciated by one of ordinary skill in the art that the hydrotreated product in conduit 708 and the hydrodeoxygenated product in conduit 725 can be combined to undergo a combined separation (as in FIGS. 5-6), FIG. 7 shows that these streams can be separated individually. One reason for individualizing the separations of these products can be the enhanced corrosive nature (e.g., linked to the carbon oxide, or specifically CO₂, content) of the hydrodeoxygenated product in conduit 725, relative to the hydrotreated product in conduit 708.

In FIG. 7, hydrodeoxygenated product in conduit 725 can be sent to a (hot) biocomponent separator drum 735 to obtain a (hot) biocomponent vapor phase component that can exit through conduit 734, optionally a (hot) biocomponent aqueous phase (not shown), and a (hot) biocomponent liquid phase component that can exit through conduit 736. At least a portion of the separated liquid phase component can advantageously be recycled through conduit 738. Like in FIG. 6, this recycle first goes through a hydroisomerization reactor 730, which contains a hydroisomerization catalyst (e.g., a zeolite-supported catalyst containing a noble Group VIII metal or a non-noble Group VIII metal and a Group VIB metal, generally sulfided, such as a Pt- and/or Pd-promoted zeolite Y dewaxing catalyst, a Pt-promoted ZSM-48 dewaxing catalyst, and/or a NiW-promoted ZSM-48 dewaxing catalyst) and is fed with a hydrogen source 722. Recycling the dewaxed/isomerized product through conduit 732 can allow a feed to be further hydrodeoxygenated, to have advantageously better cold-flow properties (e.g., lower relative cloud point, lower relative pour point, lower relative cold filter plugging point, or the like, or a combination thereof) for managing the tendency of hydrodeoxygenated biocomponent products to have relatively poor (e.g., off-spec high) cold-flow properties, and/or to act as a diluent to manage the large exotherms common in biocomponent hydrodeoxygenation reactions. Optionally but preferably, another portion of the separated liquid phase component, which has sufficiently low oxygen, nitrogen, and sulfur content, can be sent to the fuel pool, e.g., as diesel fuel, through conduit 764. Though not pictured this way in FIG. 7, this other portion of the separated liquid phase component may not have sufficiently low heteroatom levels, in which case it can optionally first be sent to stripper 760 before being directed to the fuel pool through conduit 762.

In FIG. 7, hydrotreated product in conduit 708 can be sent to a hot mineral separator drum 740 to obtain a hot mineral vapor phase component that can exit through conduit 742, optionally a hot mineral aqueous phase (not shown), and a hot mineral liquid phase component that can exit through conduit 744. At least a portion of the separated liquid phase component can advantageously be recycled through conduit 748, where it can be combined with the separated liquid phase component from the (hot) biocomponent separator in conduit 738 to form combined liquid phase component in conduit 758 for recycle via hydroisomerization reactor 730. Optionally but preferably, another portion of the separated hot liquid phase component, which has sufficiently low oxygen, nitrogen, and sulfur content, can be sent to the fuel pool, e.g., as diesel fuel, through conduit 762. Though not pictured this way in FIG. 7, this other portion of the separated liquid phase component may not have sufficiently low heteroatom levels, in which case it can optionally first be sent to stripper 660 before being directed to the fuel pool through conduit 762. In FIG. 7, however, this other portion of the separated hot liquid phase component may not have sufficiently low heteroatom levels, in which case it is optionally but preferably first sent through conduit 746 to stripper 760 before being directed to the fuel pool through conduit 762.

While it is shown in FIG. 7 that hydrogen sources 702, 712, and 722 for reactors 705, 720, and 730, respectively, are independent, they can instead be drawn from the same source, e.g., a refinery distribution network hydrogen source.

FIG. 7 shows the mineral vapor phase component that exits hot mineral separator drum 740 through conduit 742 being directed to a cold separator drum 750, which itself can create a cold vapor phase component that can exit through conduit 752, optionally a cold aqueous phase (not shown), and a cold liquid phase component that can exit through conduit 754. A portion (though none in FIG. 7) of the cold separated liquid phase component, which has sufficiently low oxygen, nitrogen, and sulfur content, can be sent to the fuel pool, e.g., as diesel fuel, through conduit 762. As with the hot separated mineral liquid phase component, in FIG. 7, the cold separated liquid phase component may not have sufficiently low heteroatom levels, in which case it is optionally but preferably first sent through conduit 756 (where it is combined with the hot separated mineral liquid phase component from conduit 746) to stripper 760 before being directed to the fuel pool through conduit 762.

The biocomponent (hot) vapor phase component in conduit 734 and the cold vapor phase component in conduit 752, independently or collectively can be: sent to flare; used as fuel gas; purified to isolate one or more higher value components (such as hydrogen) therefrom; recycled and/or cascaded to the hydrodeoxygenation reactor 720, the hydrotreatment reactor 705, the hydroisomerization reactor 730, and/or another refinery process needing hydrogen or another gaseous component; or the like; or some combination thereof. Although FIG. 7 shows a three-stage separation using (hot) biocomponent separator drum 735, hot mineral separator drum 740, and cold separator drum 750, it is of course contemplated that more than three separation stages may be used, or alternately that less than three separation stages may be used. In one alternative embodiment, where there is only (hot) biocomponent separator drum 735 and hot mineral separator drum 740, the vapor phase component 742 from sole mineral separator drum 740 can be disposed of as detailed for biocomponent (hot) vapor phase component in conduit 734 and/or cold vapor phase component in conduit 752 above, with cold separator 750 and conduits 752 and 754 being removed from FIG. 7.

Furthermore, though the (hot) biocomponent liquid phase recycle portion and the hot mineral liquid phase recycle portion are shown in FIG. 7 as being individually linked into the recycle conduit upstream/downstream from each other and as being separated from the portion (hot) biocomponent and hot mineral liquid phase portions, respectively, that are being sent to the fuel pool, it should be understood that the streams may be combined in the recycle conduit in any order or together and that the streams may additionally or alternately be combined to be sent to the fuel pool, optionally through stripper 760 as needed.

### Additional Embodiments

The following embodiments can additionally or alternately be included in the invention as follows.

### Embodiment 1

A method for forming a biocomponent-containing diesel boiling range product, comprising: hydrodeoxygenating a feedstock comprised of about 50 wt % or less of a fresh biocomponent portion and having an oxygen content of at least 2 wt % by contacting the feedstock with a treat gas comprising at least about 80 mol % hydrogen in the presence of a hydrodeoxygenation catalyst under hydrodeoxygenation conditions to form a hydrodeoxygenated product; and separating the hydrodeoxygenated product into a liquid effluent and a vapor effluent, at least a first portion of the liquid effluent having an oxygen content of about 500 wppm or less being recycled to the to hydrodeoxygenation step, and at least a second portion of the liquid effluent having an oxygen content of about 500 wppm or less being a diesel boiling range product, wherein the hydrodeoxygenation conditions comprise a total pressure from about 80 psig (about 0.5 MPag) to about 400 psig (about 2.8 MPag), and wherein the hydrodeoxygenation catalyst comprises a catalyst having an activity that is about 50% or less of an activity of a fresh hydrotreating catalyst having hydrodenitrogenation and/or hydrodesulfurization functionality.

### Embodiment 2

The method of embodiment 1, wherein the hydrodeoxygenation catalyst comprises a water gas shift catalyst, for example comprising an oxide of iron, copper, zinc, chromium, or a combination thereof.

### Embodiment 3

The method of embodiment 2, wherein the water gas shift catalyst comprises Fe₃O₄.

### Embodiment 4

The method of any one of the previous embodiments, wherein the catalyst comprises a spent hydrotreating catalyst.

### Embodiment 5

The method of embodiment 4, wherein the spent hydrotreating catalyst comprises at least one metal from Group VIB of the Periodic Table of Elements and at least one metal from Group VIII of the Periodic Table of Elements, for example comprising Ni and/or Co and Mo and/or W.

### Embodiment 6

The method of any one of the previous embodiments, wherein the vapor effluent is used as a refinery fuel gas.

### Embodiment 7

The method of any one of the previous embodiments, further comprising hydroisomerizing the first portion of the liquid effluent prior to recycle in the presence of a hydroisomerization catalyst, e.g., which comprises a molecular sieve having a SiO₂:Al₂O₃ ratio of 100 or less, and a metal oxide binder, wherein the hydroisomerization catalyst has a ratio of zeolite surface area to external surface area of at least 80:100.

### Embodiment 8

The method of embodiment 7, wherein one or more of the following are satisfied: the molecular sieve is EU-1, zeolite beta, ZSM-35, ZSM-11, ZSM-57, NU-87, ZSM-22, EU-2, EU-11, ZBM-30, ZSM-48, ZSM-23, or a combination thereof; the metal oxide binder in powder form has a surface area of 100 m²/g or less; the metal oxide binder comprises at least one of silica, alumina, titania, and zirconia; and the hydroisomerization catalyst also comprises a promoter metal selected from the metals of Group VIII of the Periodic Table of Elements.

### Embodiment 9

The method of any one of the previous embodiments, further comprising mixing the hydrodeoxygenated product with a hydrotreated mineral hydrocarbon stream (e.g., which exhibits a diesel and/or higher temperature boiling range, such as which comprises a diesel boiling range stream, an atmospheric gasoil stream, a vacuum gasoil stream, or a combination thereof) having a sulfur content of about 30 wppm or less and a nitrogen content of about 30 wppm or less.

### Embodiment 10

The method of any one of the previous embodiments, wherein the feedstock comprises at least 5 wt % of a mineral hydrocarbon component having a sulfur content of about 100 wppm or less and a nitrogen content of about 50 wppm or less.

### Embodiment 11

The method of embodiment 10, wherein the mineral hydrocarbon component comprises a kerosene boiling range stream.

### Embodiment 12

The method of embodiment 10 or embodiment 11, wherein the feedstock has not previously been catalytically hydrotreated.

### EXAMPLES

### Example 1

A mineral ADO is hydrotreated to yield a product suitable for use in the diesel fuel pool, having a finished sulfur content of about 3 wppm or less and a finished nitrogen content of about 2 wppm or less, and exhibiting an API gravity of about 34.9 (degrees). About 70 wt % of this hydrotreated mineral ADO is added to about 30 wt % soybean oil to form a mixed feed. This mixed feed is contacted in a hydroprocessing reactor with a treat gas comprising about 40 mol % hydrogen and about 60 mol % methane in the presence of a commercial supported (alumina) NiMo hydrotreating catalyst at a temperature of about 625° F. (about 329° C.) and at a total reactor pressure of about 200 psig (about 1.4 MPag). The hydrotreating catalyst is not fresh and has been previously used to hydrotreat vacuum gas oils (VGO) to the extent that the catalyst has about a 10% activity reduction, as compared to fresh catalyst of identical composition. The treat gas is introduced at a rate of about 2250 scf/bbl (about 380 Sm³/m³). This corresponds to an equivalent hydrogen treat gas rate of about 900 scf/bbl (about 150 Sm³/m³) and a hydrogen partial pressure of about 60-80 psig (about 410−550 kPag). The hydroprocessing reactor has an LHSV of about 1.0 hr⁻¹.

The hydrotreated mixed feed yields about 95 wt % recovery, based on the original mixed feed plus hydrogen-containing treat gas, and exhibits an API gravity of about 42.2 (degrees). This represents an improvement in API of more than about 20%. Significant water, CO, and CO₂ are formed during the reaction, and some naphtha is also made (which can be sent to the gasoline pool, if desired, can be recycled to another refinery process). At least about 90% of the oxygen from the biocomponent portion of the mixed feed is removed by this process.

### Example 2

A mixed feedstock, containing about 30 wt % of a biocomponent feed (soybean oil) and about 70 wt % of a mineral feed (bottoms from a hydrocracking unit), as described in Table 1 below, was hydrodeoxygenated in a pilot unit comprising a ˜28 cm³ guard bed (diolefin saturator) loaded with a commercially-available low activity supported CoMo catalyst upstream of a ˜100 cm³ HDO reactor loaded with an activated catalyst based on a commercially available supported NiMo catalyst. The HDO catalyst was activated using a DMDS spiked light gasoil. The HDO reactor EIT was thereafter brought to about 625° F. (about 329° C.), the guard bed EIT to about 450° F. (about 232° C.), and the unit pressure to about 150 psig (about 1.0 MPag). This HDO reactor was run using a substantially pure (˜100%) hydrogen treat gas at a rate of about 900 scf/bbl (about 150 Nm³/m³), with the feed being run therethrough at an LHSV of about 1.3 hr⁻¹.

TABLE 1

| | Base Feed | | | |
|---|---|---|---|---|
| | | | Bio- | |
| | light | Mineral | component | Mixed |
| | gasoil | feed | feed | feed |
| Soybean Oil Content | — | — | 100 wt % | 30 wt % |
| Mineral Oil Content | 100 wt % | 100 wt % | — | 70 wt % |
| API gravity | 35.3 | 40.4 | 21.7 | 34.9 |
| Sulfur, wppm | 4800 | 0.35 | <0.3 | |
| Nitrogen, wppm | | <0.2 | 14 | |
| Bromine # | 1.73 | | 62.8 | ~19 |
| IBP, ° F. | 243 | 295 | | 178 |
| T5, ° F. | 385 | 352 | | 362 |
| T10, ° F. | 437 | 380 | | 395 |
| T20, ° F. | 483 | 417 | | 436 |
| T30, ° F. | 508 | 442 | | 469 |
| T40, ° F. | 525 | 466 | | 509 |
| T50, ° F. | 544 | 493 | | 560 |
| T60, ° F. | 561 | 528 | | 612 |
| T70, ° F. | 577 | 566 | | 690 |
| T80, ° F. | 595 | 600 | | 1111 |
| T90, ° F. | 615 | 655 | | 1125 |
| T95, ° F. | 628 | 689 | | 1127 |
| T99.5, ° F. | 654 | 763 | | 1133 |
| 1-Ring Aromatics | 20.0 wt % | 15.5 wt % | | 10.8 wt % |
| 2-Ring Aromatics | 8.2 wt % | 1.3 wt % | | 1.0 wt % |
| 3-Ring Aromatics | 0.4 wt % | 0.1 wt % | | 0.2 wt % |
| Total Aromatics | 28.6 wt % | 17.0 wt % | | 11.9 wt % |
| H₂ Content, mass % | 13.2 | 13.9 | | |

The sample was run for about 4 days on oil, and the liquid product was periodically sampled. After about 4 days, the liquid product exhibited: a color of about 1.8, when measured according to AS™ D1500-1; a total aromatics content of about 13 mass %, with a 2-ring aromatics content of about 2.2 wt %; a bromine number of about 0.3; a CO content in exit gas (on the basis of carbon-containing gases and hydrogen gas, i.e., on a nitrogen-free, oxygen-free, ammonia-free, and H₂S-free basis) of about 4.1 mol %, which corresponds to a CO yield from the biocomponent portion of about 2.0 wt %; and a CO₂ content in exit gas (on the basis of carbon-containing gases and hydrogen gas, i.e., on a nitrogen-free, oxygen-free, ammonia-free, and H₂S-free basis) of about 8.9 mol %, which corresponds to a CO₂ yield from the biocomponent portion of about 6.9 wt %. The hydrogen consumption for the deoxygenation reaction was calculated to be from about 1500 scf/bbl (about 250 Nm³/m³) to about 1600 scf/bbl (about 270 Nm³/m³). The liquid phase product of the reaction was also analyzed to determine the relative amounts of the different carbon number products, as follows: about 1.9 mass % C₁₂ isomers; about 2.1 mass % C₁₃ isomers; about 2.3 mass % C₁₄ isomers; about 1.5 mass % C₁₅ isomers; about 7.2 mass % n-C₁₅; about 0.9 mass % C₁₆ isomers; about 2.7 mass % n-C₁₆; about 2.5 mass % C₁₇ isomers; about 51.6 mass % n-C₁₇; about 1.0 mass % C₁₈ isomers; about 19.4 mass % n-C₂₀; about 0.4 mass % C₁₉ isomers; about 3.4 mass % C₂₀ isomers; about 0.5 mass % n-C₂₀; and about 2.4 mass % C₂₁₊ species.

### Examples 3-5

A mixed feedstock, containing about 50 wt % of a biocomponent feed (soybean oil) and about 50 wt % of a mineral feed (bottoms from a hydrocracking unit), as described above in Table 1, was hydrodeoxygenated in several parallel pilot units comprising a ˜10 cm³ HDO reactor loaded with an activated catalyst based on a commercially available supported NiMo catalyst, and a ˜7 cm³ HDO reactor loaded with an activated bulk NiMo catalyst. Both catalysts were activated using a DMDS spiked light gasoil. Each reactor EIT was thereafter brought to about 590° F. (about 310° C.), and each unit pressure to about 300 psig (about 2.1 MPag) (Example 3) or about 150 psig (about 1.0 MPag) (Examples 4-5). Each unit was run using a substantially pure (˜100%) hydrogen treat gas at a rate of about 1200 scf/bbl (about 200 Nm³/m³) (Examples 3-5) or about 1050 scf/bbl (about 175 Nm³/m³) (Example 5 was run with the supported NiMo at 1200 scf/bbl while the bulk NiMo was run at 1050 scf/bbl treat gas rate), with the feed being run through each unit at an LHSV of about 1 hr⁻¹ (Examples 3-4, supported NiMo), about 2 hr⁻¹ (Example 5, supported NiMo), about 1.4 hr⁻¹ (Examples 3-4, bulk NiMo), or about 2.9 hr⁻¹ (Example 5, bulk NiMo).

Each sample was run for about 2 days on oil, with the supported NiMo catalyst having a relative activity of about 50%, compared to the fresh catalyst activity (in this case, of the fresh HDS activity), and with the bulk NiMo catalyst having a relative activity of about 40%, compared to the fresh catalyst activity. The liquid product was periodically sampled. After about 2 days, the liquid products had the properties listed in Table 2. The hydrogen consumption for the reaction was calculated assuming no methanation reactions occur.

TABLE 2

| | Example 3 | | Example 4 | | Example 5 | |
|---|---|---|---|---|---|---|
| | Supported | | Supported | | Supported | |
| | NiMo | Bulk NiMo | NiMo | Bulk NiMo | NiMo | Bulk NiMo |
| % fresh HDS activity | 50 | 40 | 50 | 40 | 50 | 40 |
| EIT, ° F. | 590 | 590 | 590 | 590 | 590 | 590 |
| Total pressure, psig | 300 | 300 | 150 | 150 | 150 | 150 |
| LHSV, hr⁻¹ | 1 | 1.4 | 1 | 1.4 | 2 | 2.9 |
| H₂ purity, mol % | ~100 | ~100 | ~100 | ~100 | ~100 | ~100 |
| Treat gas rate, scf/bbl | 1200 | 1200 | 1200 | 1200 | 1200 | 1050 |
| CO in exit gas, mol % | 3.9 | 6.6 | 7.7 | 9.9 | 12.0 | 18.0 |
| CO₂ in exit gas, mol % | 10.9 | 8.9 | 6.1 | 5.3 | 6.1 | 5.3 |
| CO yield on soy oil, | 2.1 | 4.3 | 4.1 | 6.5 | 7.1 | 8.2 |
| wt % | | | | | | |
| CO₂ yield on soy oil, | 5.7 | 5.9 | 3.6 | 2.4 | 3.6 | 2.4 |
| wt % | | | | | | |
| CH₄ yield on soy oil, | 0.08 | 0.07 | 0.07 | 0.06 | 0.08 | 0.05 |
| wt % | | | | | | |
| H₂ Consumption, | 1593 | 1393 | 1528 | 1368 | 1338 | 1342 |
| scf/bbl (calc.) | | | | | | |
| C₁₂ content, mass % | — | — | 3.5 | — | 3.2 | 1.4 |
| C₁₃ content, mass % | — | — | 0.9 | — | 2.4 | 0.8 |
| C₁₄ content, mass % | — | — | 2.2 | — | 2.4 | 2.0 |
| C₁₅ content, mass % | — | — | 8.4 | — | 7.7 | 8.6 |
| C₁₆ content, mass % | — | — | 4.6 | — | 2.2 | 1.2 |
| C₁₇ content, mass % | — | — | 50.8 | — | 58.2 | 64.0 |
| C₁₈ content, mass % | — | — | 14.4 | — | 17.9 | 11.2 |
| C₁₉ content, mass % | — | — | 1.0 | — | 0.8 | 0.6 |
| C₂₀ content, mass % | — | — | 2.9 | — | 0.5 | 0.9 |
| C₂₁ content, mass % | — | — | 2.4 | — | 0.7 | 0.7 |
| C₂₂ content, mass % | — | — | 1.9 | — | 0.4 | 0.4 |
| C₂₃ content, mass % | — | — | 2.3 | — | 1.3 | 1.1 |
| C₂₄ content, mass % | — | — | 1.7 | — | 0.2 | 1.1 |
| C₂₅ content, mass % | — | — | 1.3 | — | 0.2 | 1.3 |
| C₂₆ content, mass % | — | — | 0.9 | — | 0.4 | 1.2 |
| C₂₇ content, mass % | — | — | 0.5 | — | 0.0 | 0.9 |
| C₂₈ content, mass % | — | — | 0.2 | — | 0.0 | 0.7 |
| C₂₉ content, mass % | — | — | 0.1 | — | 0.0 | 0.6 |
| Oxygenate content, | — | — | 0.2 | — | 1.7 | 1.4 |
| mass % | | | | | | |

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

What is claimed is:

1. A method for forming a biocomponent-containing diesel boiling range product, comprising:
hydrodeoxygenating a feedstock comprised of about 50 wt % or less of a fresh biocomponent portion and having an oxygen content of at least 2 wt % by contacting the feedstock with a treat gas comprising at least about 80 mol % hydrogen in the presence of a hydrodeoxygenation catalyst under hydrodeoxygenation conditions to form a hydrodeoxygenated product; and
separating the hydrodeoxygenated product into a liquid effluent and a vapor effluent, at least a first portion of the liquid effluent having an oxygen content of about 500 wppm or less being recycled to the hydrodeoxygenation step, and at least a second portion of the liquid effluent having an oxygen content of about 500 wppm or less being a diesel boiling range product,
wherein the hydrodeoxygenation conditions comprise a total pressure from about 80 psig (about 0.5 MPag) to about 400 psig (about 2.8 MPag), and wherein the hydrodeoxygenation catalyst comprises a catalyst having an activity that is about 50% or less of an activity of a fresh hydrotreating catalyst having hydrodenitrogenation and/or hydrodesulfurization functionality.

2. The method of claim 1, wherein the hydrodeoxygenation catalyst is a water gas shift catalyst.

3. The method of claim 2, wherein the water gas shift catalyst comprises an oxide of iron, copper, zinc, chromium, or a combination thereof.

4. The method of claim 3, wherein the water gas shift catalyst comprises Fe₃O₄.

5. The method of claim 1, wherein the catalyst comprises a spent hydrotreating catalyst.

6. The method of claim 5, wherein the spent hydrotreating catalyst comprises at least one metal from Group VIB of the Periodic Table of Elements and at least one metal from Group VIII of the Periodic Table of Elements.

7. The method of claim 6, wherein the spent hydrotreating catalyst comprises Ni and/or Co and comprises Mo and/or W.

8. The method of claim 1, wherein the vapor effluent is used as a refinery fuel gas.

9. The method of claim 1, further comprising hydroisomerizing the first portion of the liquid effluent prior to recycle.

10. The method of claim 9, wherein the hydroisomerization catalyst comprises a molecular sieve having a SiO₂:Al₂O₃ ratio of 100 or less, and a metal oxide binder, and the dewaxing catalyst has a ratio of zeolite surface area to external surface area of at least 80:100.

11. The method of claim 10, wherein one or more of the following are satisfied:
the molecular sieve is EU-1, zeolite beta, ZSM-35, ZSM-11, ZSM-57, NU-87, ZSM-22, EU-2, EU-11, ZBM-30, ZSM-48, ZSM-23, or a combination thereof;
the metal oxide binder in powder form has a surface area of 100 m²/g or less;
the metal oxide binder comprises at least one of silica, alumina, titania, and zirconia; and
the hydroisomerization catalyst also comprises a promoter metal selected from the metals of Group VIII of the Periodic Table of Elements.

12. The method of claim 1, further comprising mixing the hydrodeoxygenated product with a hydrotreated mineral hydrocarbon stream having a sulfur content of about 30 wppm or less and a nitrogen content of about 30 wppm or less.

13. The method of claim 12, wherein the hydrotreated mineral hydrocarbon stream is in a diesel and/or higher temperature boiling range.

14. The method of claim 1, wherein the feedstock comprises at least 5 wt % of a mineral hydrocarbon component having a sulfur content of about 100 wppm or less and a nitrogen content of about 50 wppm or less.

15. The method of claim 14, wherein the mineral hydrocarbon component comprises a kerosene boiling range stream.

16. The method of claim 14, wherein the feedstock has not previously been catalytically hydrotreated.
